(19) United States
(12) Patent Application Publication
(10) Pub. No.: US 2022/0071233 A1
(43) Pub. Date: Mar. 10, 2022

(54) IN VITRO INSECT MUSCLE AS A NUTRITION SOURCE

(71) Applicant: Trustees of Tufts College, Medford, MA (US)

(72) Inventors: David L. Kaplan, Concord, MA (US); Natalie Rubio, Medford, MA (US)

(21) Appl. No.: 17/309,816

(22) PCT Filed: Dec. 16, 2019
(86) PCT No.: PCT/US19/66452

§ 371 Date: Jun. 21, 2021

Related U.S. Application Data
(60) Provisional application No. 62/784,260, filed on Dec. 21, 2018.

(51) Int. Cl. A23J 3/34; A23J 3/22; C12N 5/07

(52) U.S. Cl. CPC A23J 3/341; A23J 3/227; C12N 2500/50; C12N 2533/72; C12N 2500/24; C12N 5/0601

(57) ABSTRACT

Provided herein is a cultured meat product comprising a confluent serum-free insect muscle cell culture seeded on a food safe substrate. Further provided herein is a method for producing a cultured meat product comprising the steps of: culturing insect muscle cells on a food safe substrate in serum-free culture medium for a time sufficient for the cells to reach confluence. Also provided herein is a bioactuator comprising confluent insect muscle cells cultured in a flexible substrate to form muscle fibers.

### CROSS-REFERENCE TO RELATED APPLICATIONS

This application claims priority to U.S. Provisional Application No. 62/784,260, filed Dec. 21, 2018, which is incorporated herein in its entirety.

### STATEMENT REGARDING FEDERALLY SPONSORED RESEARCH

N/A

### BACKGROUND

Skeletal muscle tissue engineering research typically utilizes mammalian model cell lines such as the mouse myoblast cell line C2C12, the rat myoblast cell line L6 or human cells obtained from primary lines or induced pluripotent stem cells. The culture conditions for these cell types are similar; growth medium typically consists of sodium bicarbonate buffered basal medium supplemented with fetal bovine serum, incubation is set at 5% carbon dioxide and 37° C., and cells are detached from their substrates via enzymatic treatment. These culture conditions, while feasible at bench-scale, create hurdles for achieving cost-efficient large-scale production when considering commercialization of cell-based products. Specifically, animal serum is costly and batch-to-batch fluctuations contribute to variability, above ambient incubation temperatures rely on increased energy use and cost, and adherent cell lines require microcarriers to attain high density. For applications of tissue engineering that do not necessitate relevance to human physiology, like cultured meat production and soft robotics (e.g., bioactuators), it is worthwhile to expand research beyond mammalian cell types.

### SUMMARY OF THE INVENTION

In a first aspect, provided herein is a cultured meat product comprising a confluent serum-free insect muscle cell culture seeded on a food safe substrate. In some embodiments, the substrate is a film. In some embodiments, the substrate is a sponge. In some embodiments, the substrate is a chitosan substrate. In some embodiments, the chitosan substrate is a mushroom chitosan substrate.

In some embodiments, the insect muscle cells in the cultured meat product are Drosophila melanogaster cells. In some embodiments, the insect muscle cells are multinucleated myotubes derived from Drosophila adult muscle progenitor cells (DrAMPCs). In some embodiments the DrAMPC derived multinucleated myotubes express myosin heavy chain and ecdysone receptor. In some embodiments, the DrAMPC derived multinucleated myotubes are produced by a method comprising: culturing a population of DrAMPCs in suspension in serum-free culture medium comprising 20-hydroxyecdysone. The serum-free culture medium may additionally comprise dextran sulfate and methoprene.

In a second aspect, provided herein is a method for producing a cultured meat product comprising the steps of: culturing insect muscle cells on a food safe substrate in serum-free culture medium for a time sufficient for the cells to reach confluence. In some embodiments, the insect muscle cells are DrAMPC derived multinucleated myotubes. In some embodiments, the DrAMPC derived multinucleated myotubes express myosin heavy chain and ecdysone receptor. In some embodiments, the DrAMPC derived multinucleated myotubes are produced by a method comprising: culturing a population of DrAMPCs in suspension in serum-free culture medium comprising 20-hydroxyecdysone. The serum-free culture medium may additionally comprise methoprene. The serum-free culture medium may additionally comprise dextran sulfate. In some embodiments, the substrate is a film or a sponge. In some embodiments, the substrate is a chitosan substrate. In some embodiments, the chitosan substrate is a mushroom chitosan substrate. In some embodiments, the confluent insect cell layer is mechanically dissociated from the substrate.

In some embodiments, prior to culturing in suspension, the DrAMPCs are cultured in serum-free culture medium on an adherent substrate. In some embodiments, the adherent substrate is a plasma-treated substrate. In some embodiments, the DrAMPCs are seeded on the adherent substrate at a density between about 55,000 cells/mL and about 95,000 cells/ml. In some embodiments, prior to culturing in serum-free culture medium, the DrAMPCs are cultured in culture medium supplemented with serum for at least about 2 days. The serum may be fetal bovine serum.

In a third aspect, provided herein is a bioactuator comprising confluent insect muscle cells cultured in a flexible substrate to form muscle fibers, wherein a first end of the insect muscle fiber is attached to a first anchor and a second end of the insect muscle fiber is attached to a second anchor; at least one electrode contacting the flexible substrate; and a culture medium contacting the flexible substrate and muscle fibers. In some embodiments, the substrate is a chitosan substrate. In some embodiments, the substrate is a chitosan sponge. In some embodiments, the substrate is a polydimethylsiloxane (PDMS) substrate. In some embodiments, the culture medium comprises potassium. In some embodiments, the insect muscle cells are DrAMPCs.

### BRIEF DESCRIPTION OF DRAWINGS

FIGS. 1A-1H show insect muscle cell adaptation to serum-free medium and transition from monolayer to single-cell suspension culture. Fluorescent microscopy images of GFP-expressing DrAMPCs which were (FIG. 1A) cultured with basal media supplemented with 10% FBS (FIG. 1B) immediately transferred from serum-supplemented media to EC405 or (FIG. 1C) gradually adapted from serum-supplemented media to increasing concentrations of EC405 over the course of two weeks. (FIG. 1D) Growth curve of DrAMPCs cultured in control media or EC405 after gradual adaptation. The cell population was quantified from Fiji image analysis and is displayed relative to the cell population as measured on Day 1. Error bars are standard deviations (n=5), and replicates are separate 24-wells. Phase contract microscopy images of DrAMPCs cultured on (FIG. 1E) plasma-treated culture surface or (FIG. 1F) ultra-low attachment surface. Fluorescent microscopy images of DrAMPCs cultured in agitated suspension in (FIG. 1G) EC405 or (FIG. 1H) EC405 supplemented with 100 μg/mL dextran sulfate.

FIGS. 2A-2C show a comparison of insect and mammalian muscle cell survival and growth during long-term absence of media refreshment. (FIG. 2A) Change in C2C12 and DrAMPC populations relative to the populations determined at Day 5. Populations were quantified from Fiji image analysis. Error bars are standard deviations (n=6); replicates are separate images. (FIG. 2B) Cell viability of C2C12 and DrAMPC populations as quantified from Fiji analysis of Live/Dead stained images. Error bars are standard deviations (n=6); replicates are separate images. Statistical significance was determined via the Holm-Sidak method, with alpha=0.05. (FIG. 2C) Fluorescence microscopy images of Live/Dead stained C2C12 and DrAMPC populations over time. White arrows on Day 25 indicate the cellular aggregates detached from the culture surface.

FIGS. 3A-3E show the effect of insect juvenile and molting hormones on in vitro proliferation and differentiation of insect muscle cells. (FIG. 3A) Cell population after treatment with 0, 500 or 1000 ng/mL of methoprene as measured via a CyQuant proliferation assay. Error bars are standard deviations (n=5); replicates are separate 96-wells. Statistical significance was determined via the Holm-Sidak method, with alpha=0.05. (FIG. 3B) Histogram of cell lengths of cultures treated with 0 or 500 ng/mL JH as measured via Fiji image analysis. (FIG. 3C) Cell population after treatment with 0, 500 or 1000 ng/mL of 20-HE as measured via a CyQuant proliferation assay. Error bars are standard deviations (n=5). Differences between averages were not statistically significant. (FIG. 3D) Fluorescence microscopy images of DrAMPCs treated with 0 or 500 ng/mL JH after 4 days in culture. Circles indicate elongated cells. (FIG. 3E) Fluorescence microscopy images of DrAMPCs treated with 40 ng/mL 20-HE.

FIGS. 4A-4C shows confirmation of muscle identity and cellular contractions induced by extracellular potassium. (FIG. 4A) Confocal microscopy image of DrAMPCs immunostained for EcdR and DAPI. (FIG. 4B) Fluorescent microscopy image of DrAMPCs cultured in EC405 and treated with 1 μg/mL 20-HE for 5 days and immunostained for myosin heavy chain. (FIG. 4C) Phase contrast images of DrAMPCs before and immediately after treatment with 600 mM K⁺ supplemented media. Circles indicate myoblasts contracting upon K⁺ treatment.

FIGS. 5A-5E show adhesion and growth of insect muscle cells on mushroom chitosan films. (FIG. 5A) SEM images of the middle and edge morphology of a 2% chitosan film. Films of other concentrations were similar in appearance. (FIG. 5B) Fluorescence microscopy images of DrAMPCs at medium (75,000 cells/cm²) and high (300,000 cells/cm²) seeding densities on chitosan films of variable concentrations after 24 hours in culture. (FIG. 5C) Viable cells on chitosan films or control conditions quantified via MTS assay. Films and controls were assayed after 24 hours in culture (initial seeding density of 300,000 cells/cm²) as they were (Total) or after being aspirated and rinsed with PBS (Adherent). (FIG. 5D) Percentage of adherent, viable cells determined via MTS assay on untreated or 0.1% gelatin coated films or controls. (FIG. 5E) Viable, adherent cells determined via MTS assay quantified after 24 hours and on day 5 of culture.

FIGS. 6A-6E show adhesion and differentiation of insect muscle cells on chitosan sponge scaffolds. (FIG. 6A) SEM images of cross-section and lateral views of a 2% chitosan sponge. Sponges of other concentrations were similar in appearance. (FIG. 6B) Confocal images of DrAMPCs on chitosan sponges of variable concentrations after 1 week in culture in control media. (FIG. 6C) Fluorescence microscopy images of chitosan sponges with and without hormone treatment. (FIG. 6D) Fluorescence microscopy image of DrAMPCs on a 4% chitosan sponge after 10 days of growth in EC405 media. The white arrow indicates the orientation of the aligned microtubular pore. (FIG. 6E) Elastic moduli of chitosan sponges obtained via hydrated compression tests performed in either the cross-sectional or lateral direction (error bars are standard deviations, n=5). The schematic demonstrates the direction of compression.

FIGS. 7A-7C show nutrient profile comparison between edible insects and conventional meat as well as cultured insect and mammalian cells. (FIG. 7A) Ranges of nutrients reported for insect species organized by insect order and compared to nutrition values of mammalian meat. (FIG. 7B) Mineral profiles of fruit flies versus beef as measured in % daily recommended value. (FIG. 7C) Assayed intracellular iron and zinc content of C2C12s and DrAMPCs.

FIG. 7D shows representative schematics for bioactuator fabrication and cultured meat production.

FIG. 8 shows directional freezing of mushroom chitosan for fabrication of sponges with aligned microtubular pores.

FIG. 9 shows fluorescent images of cells on uncoated films or films coated with 0.1% gelatin after 1 day in culture.

FIG. 10 shows thermogravimetric analysis of thermal degradation of a 2% chitosan sponge heated from room temperature to 500° C.

FIG. 11 shows confocal z-stack images of DrAMPCs on chitosan sponges after 2 weeks in culture.

FIG. 12 shows phase contract image of DrAMPCs in long-term starvation culture after more than 50 days without fresh media. Evaporation induced media participation in the form of crystals.

FIG. 13 shows DrAMPCs cultured in Schneider's Growth Media and 10% Fetal Bovine Serum supplemented with variable concentrations of silk sericin protein. Viability was assayed with a Live/Dead stain and microplate reader.

FIG. 14 shows DrAMPCs immunostained for Connectin after treatment with 20-hydroxyecdysone.

FIG. 15 shows image analysis fluorescence quantification of Connectin expression in cells untreated or treated with 20-hydroxyecdysone.

FIG. 16 shows DrAMPCs immunostained for Neuroglian after treatment with 20-hydroxyecdysone.

FIG. 17 shows image analysis fluorescence quantification of Neuroglian expression in cells untreated or treated with 20-hydroxyecdysone.

FIG. 18 shows image analysis fluorescence quantification of myosin heavy chain expression in cells untreated or treated with 20-hydroxyecdysone.

FIG. 19 shows image analysis fluorescence quantification of DAPI in cells untreated or treated with 20-hydroxyecdysone.

FIG. 20 shows Ms-EPCs isolated from eggs and cultured with or without poly-lysine and with or without rinsing off non-adherent cells 2 hours after plating. Scale bar is 100 um.

FIG. 21 shows Ms-EPCs at day 6 after being cultured on poly-lysine and differentiated with 20-hydroxyecdysone and stained for myosin heavy chain and DAPI.

FIG. 22 shows BG2 cells stained with phalloidin and DAPI.

FIG. 23 shows BG2 and DrAMPC co-culture.

### DETAILED DESCRIPTION OF THE INVENTION

All publications, including but not limited to patents and patent applications, cited in this specification are herein incorporated by reference as though set forth in their entirety in the present application.

Insect cells in particular are a promising platform for tissue engineering due to simple isolation and maintenance methods and existing infrastructure for industrial-scale production. Applications of insect cell, and particularly insect muscle cell, tissue engineering described herein include insect muscle cell tissue engineered bioactuators and insect muscle cell cultured meat products. Dissimilar to medical applications, these goals are not constrained by concerns of immunogenicity, host-integration or in vivo-like function. Instead, design considerations for these technologies include large-scale, low-cost production in order for tissue-engineered commodities to be competitive against their conventional counterparts (e.g., electrical actuators, farmed meat). Bioactuators should also generate large contractile force, operate under a range of environmental conditions and incorporate control systems. In contrast, muscle produced for food applications does not need to function mechanically, but instead should be visually identical to farmed meat, palatable to consumers and provide nutritional benefit. Insect cells are better suited than mammalian cells to fulfill many of these objectives.

The present disclosure describes scaffold supported muscle growth from insect cells for production of a cultured meat product or for use in a tissue-engineered bioactuator. Insect muscle cells are fabricated into films or three-dimensional muscle tissues by culturing to confluence on a food-safe substrate or surface. Insect muscle cells for use in scaffold supported muscle growth are generated in adherent or suspension culture under serum-free conditions to differentiate insect muscle progenitor-like cells into insect muscle cells. In some embodiments, prior to culturing in suspension, the insect muscle progenitor-like cells may be cultured as a monolayer in the presence of serum.

Tissue engineered bioactuators as described herein couple cultured cells, and more specifically cultured insect muscle cells, with scaffold systems to perform mechanical work. The bioactuator is a machine that performs mechanical work and is powered by muscle fibers grown from insect muscle cells. The bioactuator includes confluent insect muscle cells cultured in a flexible substrate to form muscle fibers. A first end of the insect muscle fibers is attached to a first anchor and a second end of the insect muscle fibers is attached to a second anchor. Either or both of the first and second anchors may be mobile or repositionable. Either or both of the first and second anchors may be stationary or in a fixed position. Electrodes are connected to the flexible substrate to stimulate the muscle fibers. Upon introduction of an electrical stimuli to the flexible substrate via the electrodes, the muscle fibers will contract. The tissue engineered bioactuator may also include a culture medium in contact with or surrounding the flexible substrate and the muscle fibers. In some embodiments, the culture medium is serum-free. In some embodiments, the culture medium is supplemented with potassium. In some embodiments, the culture medium is supplemented with at least about 200 mM potassium, at least about 300 mM potassium, at least about 400 mM potassium, at least about 500 mM potassium, or at least about 600 mM potassium. The flexible substrate can include, but is not limited to polydimethylsiloxane (PDMS), chitosan, cellulose, silk, polyethylene glycol (PEG), hydrogels (e.g., chitosan, silk or cellulose hydrogels). In some embodiments, the flexible substrate is a polydimethylsiloxane (PDMS) substrate. In some embodiments, the flexible substrate is a chitosan substrate. Examples of bioactuators using mammalian cardiac or skeletal muscle cells or insect dorsal vessel explants are known and described in the art. See for example Tanaka et al. (Tanaka, Y. et al. An actuated pump on-chip powered by cultured cardiomyocytes. Lab Chip 6, 362 (2006)), Sakar et al. (Sakar, M. S. et al. Formation and optogenetic control of engineered 3D skeletal muscle bioactuators. Lab Chip 12, 4976-85 (2012)), Akiyama et al. (Akiyama, Y., Odaira, K., Iwabuchi, K. & Morishima, K. Long-term and room temperature operable bio-microrobot powered by insect heart tissue. Proc. IEEE Int. Conf. Micro Electro Mech. Syst. 145-148 (2011) doi:10.1109/MEMSYS.2011.5734382), and Baryshyan et al. (Baryshyan, A. L., Domigan, L. J., Hunt, B., Trimmer, B. A. & Kaplan, D. A. Self-assembled insect muscle bioactuators with long term function under a range of environmental conditions. R. Soc. Chem. Adv. 75, 39962-39968 (2014)), each of which is incorporated herein by reference. In some embodiments, the insect cells may be incorporated into hydrogels for 3D printing.

As used herein, “cultured meat products” refers to an edible meat product produced from cell cultures, rather than whole organisms. In generally, the cultured meat product is visually identical to farmed meat, palatable to consumers and provides nutritional benefit. Cultured meat products described herein include serum-free insect muscle cell cultures seeded on an edible, food safe substrate and cultured to confluence. The serum-free insect muscle cell culture are seeded on the food safe substrate at a density between about 20,000 cell/cm² and about 400,000 cell/cm², between about 30,000 cells/cm² and about 350,000 cells/cm², or between about 35,000 cells/cm² and about 300,000 cells/cm². In some embodiments, the cells are seeded at a density of about 50,000 cells/cm². In some embodiments, the cells are seeded at a density of about 40,000 cells/cm², about 50,000 cells/cm², about 60,000 cells/cm², about 70,000 cells/cm², about 80,000 cells/cm², about 100,000 cells/cm², about 150,000 cells/cm², about 200,000 cells/cm², about 250,000 cells/cm², about 300,000 cells/cm² or about 350,000 cells/cm². In some embodiments, the insect muscle cells become non-adherent on the food safe substrate once they reach confluence and lift off the food safe substrate without enzymatic dissociation. In some embodiments, the edible, food safe substrate is in the form of a two-dimensional film. In some embodiments, the edible, food safe substrate is in the form of a three-dimensional sponge, and the insect muscle cells form continuous muscle fibers when cultured in the sponge substrate. Suitable edible, food safe substrates are known in the art and include, but are not limited to, chitosan substrates, cellulosic substrates, silk substrates, alginate substrates, and starch substrates.

In some embodiments, the cultured meat product is produced using a chitosan substrate. The chitosan substrate can be tuned to change the adherence and growth of the insect muscle cell culture. Generally, increasing chitosan concentration in the substrate decreased adhesion of the insect muscle cells. The concentration of chitosan in the food safe substrate of the cultured meat product can be 1%, 2%, 3%, 4%, 5%, 6%, 7%, 8%, 9%, 10%, 12%, or 14%. In some embodiments, the concentration of chitosan is between about 1% and about 8%. In some embodiments, the concentration of chitosan is between about 2% and about 6%. In some embodiments, the concentration of chitosan is at least about 1%, at least about 2%, at least about 3%, at least about 4%, at least about 5%, at least about 6%, at least about 7%, or at least about 8%. Chitosan may be derived from any suitable source. Sources of chitosan include, but are not limited to, mushrooms, crustaceans, insects, green algae, and yeast. In some embodiments, the chitosan is mushroom derived chitosan.

Three-dimensional chitosan substrates or sponges suitable for use in the formation of muscle fibers or bioactuators described herein can be formed using methods known in the art. In some embodiments, chitosan sponges are formed using directional freezing of chitosan. To form chitosan sponges by directional freezing, a chitosan is dissolved in a solvent, such as, but not limited to, acetic acid, and the chitosan solution is poured into tubes. One end of the tubes is exposed to liquid nitrogen or another suitable freezing agent, such as slurry of dry ice and ethanol, until the entire solution is frozen. The frozen chitosan is then lyophilized to form the chitosan sponge. The mechanical properties of the chitosan sponge can be tuned by altering the chitosan concentration. Sponges formed using low concentration chitosan (e.g., 1%, 2%, 3% chitosan solution) have a lower elastic moduli, while sponges formed using high concentration chitosan (e.g., 6%, 7%, 8% chitosan solution) have higher elastic moduli. Chitosan may be derived from any suitable source. Sources of chitosan include, but are not limited to, mushrooms, crustaceans, insects, green algae, and yeast. In some embodiments, the chitosan is mushroom derived chitosan.

As used herein, “food safe substrate” refers to substrates that are edible or are safe for human consumption if at least a portion of the substrate remains affixed or associated with the cultured meat product. In some embodiments, the food safe substrate is a chitosan substrate. Chitosan for use as a food safe substrate may be derived from the chitin of organisms including, but not limited to, mushrooms, crustaceans, insects, green algae, and yeast. In some embodiments, the food safe substrate is a mushroom-chitosan substrate. In some embodiments, the food safe substrate is a cellulose-based substrate such as a substrate formed from decellularized plants (e.g., decellularized spinach or apples).

Cultured meat products described herein include a mineral content similar to or greater than farmed beef. In some embodiments, approximately 100 g dry weight of the cultured meat products described herein would meet the recommended daily value requirements for phosphorus, iron, zinc, manganese, and copper. In some embodiments, the cultured meat product includes Drosophila muscle cells and has an iron content of about 12.00 pg/cell (e.g., 10.50 pg/cell, 11.00 pg/cell, 11.50 pg/cell, 11.80 pg/cell, 12.00 pg/cell, 12.10 pg/cell, 12.20 pg/cell, 12.50 pg/cell, or 13.00 pg/cell). In some embodiments, the cultured meat product includes Drosophila muscle cells and has a zinc content of about 46.00 pg/cell (e.g., 44.00 pg/cell, 44.50 pg/cell, 45.00 pg/cell, 45.50 pg/cell, 45.80 pg/cell, 46.00 pg/cell, 46.10 pg/cell, 46.20 pg/cell, 46.50 pg/cell or 47.0 pg/cell). In some embodiments, the insect muscle cell culture is supplemented with iron-fortified serum and seeded on the food safe substrate to produce an iron-fortified cultured meat product. In some embodiments, the insect muscle cell culture is supplemented with zinc-fortified serum and seeded on the food safe substrate to produce zinc-fortified cultured meat product.

In some embodiments, cultured meat products described herein have a protein content between about 400 and about 1,000 pg/cell (e.g., 400 pg/cell, 450 pg/cell, 500 pg/cell, 550 pg/cell, 600 pg/cell, 650 pg/cell, 700 pg/cell, 750 pg/cell, 800 pg/cell, 850 pg/cell, 900 pg/cell, 950 pg/cell, or 1,000 pg/cell). The protein content of the culture meat products may vary depending on the size of the cells and the source of the insect muscle cells in the cultured meat product.

Serum-free insect muscle cell cultures for use in cultured meat products or bioactuators described herein can be generated using a method comprising differentiating insect muscle cells under conditions that promote differentiation of insect muscle progenitor-like cells into insect muscle cells. As used herein, the term “insect muscle cell” cells refer to insect cells of the muscle lineage obtained according to a method provided herein. Insect muscle cells can be characterized as long, multinucleated myotubes and identified by expression of myosin heavy chain and ecdysone receptor (EcdR). In some embodiments, insect muscle cells also express connectin. In some embodiments, insect cells also express neuroglian. Insect muscle cells can also be characterized by spontaneous cell contraction or by contraction following stimulation with extracellular potassium. Additionally, insect muscle cells can be characterized by growth and survival in the prolonged absence of medium refreshment. For example, the insect muscle cells can survive and grow for at least about 2 days, at least about 5 days, at least about 10 days, at least about 15 days, at least about 20 days, or at least about 25 days when the medium is not changed or supplemented after day 0. In some embodiments, the insect muscle progenitor-like cells are Drosophila melanogaster adult muscle progenitor-like cells (DrAMPCs). In some embodiments, the insect muscle cells are primary Drosophila muscle cells. In some embodiments, the insect muscle cells are primary cells from Manduca sexta embryos or adult muscles. In some embodiments, the insect muscle cells are derived from Manduca sexta embryonic precursor cells (Ms-EPC).

Differentiation of insect muscle progenitor-like cells into insect muscle cells includes culturing the insect muscle progenitor-like cells in serum-free culture medium including insect molting hormone 20-hydroxyecdysone (20-HE) for a time and under conditions suitable for cells to elongate and fuse, indicating muscle differentiation. In some embodiments, the insect muscle progenitor-like cells are cultured adhered to a substrate in serum-free culture medium including 20-HE. Insect muscle progenitor-like cell are cultured in serum-free culture medium supplemented with 20-HE for at least about 0.5 days, at least about 1 day, at least about 2 days, at least about 3 days, or at least about 5 days to form insect muscle cells expressing myosin heavy chain and EcdR. In some embodiments, differentiation of insect muscle cells is observed after about 24 ours in culture with 20-HE. 20-HE is present in the culture medium at a concentration of at least 20 ng/ml, at least about 30 ng/ml, at least about 40 ng/ml, at least about 50 ng/ml, at least about 75 ng/ml, at least about 100 ng/ml, at least about 200 ng/ml, at least about 300 ng/ml, at least about 400 ng/ml, at least about 500 ng/ml, at least about 600 ng/ml, at least about 700 ng/ml, at least about 800 mg/ml, at least about 900 ng/ml, or at least about 100 ng/ml. The 20-HE may be present in the culture medium at a concentration of between about 20 ng/ml and about 2000 ng/ml, between about 40 ng/ml and about 1500 ng/ml, between about 50 ng/ml and about 1000 ng/ml, between about 100 ng/ml and about 1000 ng/ml, or between about 100 ng/ml and about 500 ng/ml. In some embodiments, the serum-free culture medium is a commercially available culture medium such as Ex-Cell 405. The culturing can take place in any appropriate vessel (e.g., in two-dimensional plates or three-dimensional shaker flask culture). In some embodiments, the insect muscle progenitor-like cells are cultured in shaker flasks. In some embodiments, the insect muscle progenitor-like cells are cultured in a static suspension culture in ultra-low attachment plates. In some embodiments, DrAMPCs are cultured in serum-free medium supplemented with 20-HE while adhered to a substrate. In some embodiments, the insect muscle progenitor-like cells are adhered to a substrate in the presence of serum-free culture medium then supplemented with 20-HE. In some embodiments, the insect muscle progenitor-like cells are cultured in serum-free medium supplemented with 20-HE while adhered to a substrate coated with Concanavalin A, laminin, and/or poly-lysine.

In some embodiments, the insect muscle progenitor-like cells are cultured in serum-free culture medium supplemented with 20-HE and methoprene (JH). Methoprene is a juvenile hormone mimic and insecticide. The methoprene is provided in the culture medium at a concentration of at least 25 ng/ml, at least about 50 ng/ml, at least about 100 ng/ml, at least about 250 ng/ml, or at least about 500 ng/ml. The methoprene may be provided at a concentration between about 25 ng/ml and about 1000 ng/ml, between about 50 ng/ml and about 800 ng/ml, between about 100 ng/ml and about 750 ng/ml, or between about 250 ng/ml and about 600 ng/ml. The culturing can take place in any appropriate vessel (e.g., in two-dimensional plates or three-dimensional shaker flask culture). In some embodiments, DrAMPCs are cultured in serum-free medium supplemented with 20-HE and methoprene while adhered to a substrate. In some embodiments, the insect muscle progenitor-like cells are adhered to a substrate in the presence of serum-free culture medium then supplemented with 20-HE and methoprene after attachment. In some embodiments, the insect muscle progenitor-like cells are cultured in serum-free medium supplemented with 20-HE and methoprene while adhered to a substrate coated with Concanavalin A, laminin, and/or poly-lysine.

In some embodiments, the insect muscle progenitor-like cells are cultured in serum-free culture medium supplemented with 20-HE and sericin protein. The sericin protein is provided in the culture medium at a concentration of at least 5 μg/ml, at least about 10 μg/ml, at least about 15 μg/ml, at least about 20 μg/ml, at least about 30 μg/ml, at least about 50 μg/ml, at least about 75 μg/ml at least about 100 μg/ml, or at least about 120 μg/ml. The methoprene may be provided at a concentration between about 5 μg/ml and about 150 μg/ml, between about 10 μg/ml and about 120 μg/ml, between about 15 μg/ml and about 100 μg/ml, or between about 20 μg/ml and about 75 μg/ml. The culturing can take place in any appropriate vessel (e.g., in two-dimensional plates or three-dimensional shaker flask culture). In some embodiments, DrAMPCs are cultured in serum-free medium supplemented with 20-HE and sericin while adhered to a substrate. In some embodiments, the insect muscle progenitor-like cells are adhered to a substrate in the presence of serum-free culture medium then supplemented with 20-HE and sericin after attachment. In some embodiments, the insect muscle progenitor-like cells are cultured in serum-free medium supplemented with 20-HE and sericin while adhered to a substrate coated with Concanavalin A, laminin, and/or poly-lysine. In some embodiments, the insect muscle progenitor-like cells are cultured in serum-free medium supplemented with 20-HE, sericin, and methoprene.

In some embodiments, the insect muscle progenitor-like cells are cultured in serum-free culture medium supplemented with an anti-agglomeration agent, such as dextran sulfate. The dextran sulfate is provided in the culture medium at a concentration of at least 50 μg/ml, at least about 75 μg/ml, at least about 100 μg/ml, at least about 125 μg/ml, or at least about 150 μg/ml. The dextran sulfate may be provided at a concentration between about 25 μg/ml and about 200 μg/ml, between about 50 μg/ml and about 150 μg/ml or between about 75 μg/ml and about 125 μg/ml. The culturing can take place in any appropriate vessel (e.g., in two-dimensional plates or three-dimensional shaker flask culture). In some embodiments, the insect muscle progenitor-like cells are cultured in serum-free culture medium supplemented with 20-HE, methoprene, and dextran sulfate. In some embodiments, the insect muscle progenitor-like cells are cultured in serum-free culture medium supplemented with 20-HE, sericin, methoprene, and dextran sulfate. In some embodiments, DrAMPCs are cultured in suspension in serum-free medium supplemented dextran sulfate. In some embodiments, the insect muscle progenitor-like cells are cultured in serum-free medium supplemented with 20-HE, methoprene, and dextran sulfate while adhered to a substrate coated with Concanavalin A, laminin, and/or poly-lysine.

In some embodiments, the insect muscle progenitor-like cells are transitioned from adherent culture to suspension culture for expansion then transitioned back to adherent culture for differentiation to insect muscle cells. The insect muscle progenitor-like cells may be transitioned from an adherent culture surface to a non-adherent suspension culture. In some embodiments, the cells are transitions from a plasma-treated culture surface to an ultra-low attachment surface. In some embodiments, the addition of dextran sulfate to the insect cell culture transitions cells in an adherent monolayer to suspension.

In some embodiments, prior to differentiation, insect muscle progenitor-like cells are cultured under adherent conditions in the presence of serum. Insect muscle progenitor-like cells are seeded on an adherent culture surface and cultures in serum-supplemented medium. The insect muscle progenitor-like cells are seeded as a monolayer at high density. The culture medium is gradually transitioned from serum-supplemented medium to serum-free medium by reducing the concentration of serum in the medium over the course of at least about 2 days, at least about 3 days, at least about 4 days, at least about 5 days, at least about 7 days, at least about 8 days, at least about 10 days, at least about 12 days, or at least about 14. The serum-supplemented medium may be any medium known or used in the art to support the growth and proliferation of insect cells or insect muscle progenitor-like cells. In some embodiments, the serum-supplemented medium is Dulbecco Modified Eagle Medium (DMEM) or Schneider's Growth Medium, each supplemented with 10% FBS. The culturing can take place on any appropriate surface. In some embodiments, the serum-free supplemented medium is M3 Shield and Sang Medium supplemented with bactopeptone, yeast extract, and 10% FBS. In some embodiments, the cells are seeded on a plasma-treated culture surface. In some embodiments, the insect muscle progenitor-like cells are Drosophila melanogaster adult muscle progenitor-like cells (DrAMPCs). In some embodiments, the insect muscle progenitor-like cells are Manduca sexta embryonic precursor cells (Ms-EPCs).

In some embodiments of the insect muscle cell differentiation methods, insect muscle progenitor-like cells are co-cultured with insect neuronal cells. In some embodiments, the insect neuronal cells are ML-DmBG2 Drosophila melanogaster central nervous system cells (BG2). Co-culture of the insect muscle progenitor-like cells with the insect neuronal cells may help increase the rate of differentiation, increase the yield, or increase the viability or proliferation of the insect muscle cells. Insect neurons and insect muscle progenitor-like cells are adapted to the same medium formulation. In some embodiments, the medium is Schneider's+10% FBS. In some embodiments, the medium is M3 Shields & Sang supplemented with bactopeptone, yeast extract, 10% FBS and insulin. In some embodiments, the medium is Ex-Cell 405 Serum-Free insect cell medium. In some embodiments, the medium is IPL-41, hy-soy protein hydrolysate, yeast olate ultrafiltrate, lipid-sterol emulsion (See Donaldson et al. “Low-cost serum-free medium for the BTI-Tn5B1-4 insect cell line,” Biotechnol Prog., 1998, 14(4):573-579). Following medium adaptation, insect neurons are cultured on a substrate for at least 24 hours (e.g., at least 24 hours, at least 36 hours, at least 48 hours, at least 3 days, at least 4 days, at least 5 days, at least 6 days, or at least 7 days). In some embodiments, insect neuron cells are cultured on a substrate for between 2 day and 8 days. In some embodiments, insect neuron cells are cultured on a substrate for between 3 days and 7 days. Following culture of the insect cells on a substrate, insect muscle progenitor-like cells are added on top of the insect neuron cell monolayer and cultured for at least 24 hours. Following the at least 24 co-culture of the insect neuron cells with the insect muscle progenitor-like cells, a culture medium suitable for differentiating insect muscle cells in added to the co-culture. Suitable medium is described herein and may include 20-HE, sericin, methoprene, dextran sulfate, and combinations thereof.

In some embodiments, the insect muscle progenitor-like cells are Manduca sexta embryonic precursor cells (Ms-EPCs). The Ms-EPCs may be seeded on a substrate and cultured in serum-free culture medium supplemented with 20-HE. In some embodiments, the serum-free culture medium is also supplemented with sericin, methoprene, dextran sulfate, or combinations thereof. In some embodiments, the substrate is coated with poly-lysine. In some embodiments, the non-adherent cells in the Ms-EPC culture are retained (i.e., not washed away). The Manduca sexta insect muscle cells derived from the Ms-EPCs are multinucleated, striated and express myosin heavy chain.

As used herein, “high density” refers to cells seeded at a density over 160,000 cells/mL to about 480,000 cells/ml for suspension culture or over 100,000 cells/cm² to about 300,000 cells/cm² for adherent culture.

As used herein, “medium density” refers to cells seeded at a density of over 64,000 cells/ml to about 160,000 cells/ml for suspension culture or over 40,000 cells/cm² to about 100,000 cells/cm² for adherent culture.

As used herein, “low density” refers to cells seeded at a density of about 16,000 cells/ml to about 64,000 cells/ml for suspension culture or between about 10,000 cells/cm² to about 40,000 cells/cm² for adherent culture.

As used herein, “serum-free” refers to culture conditions and culture medium that does not contain serum or serum replacement, or that it contains essentially no serum or serum replacement. For example, an essentially serum-free medium can contain less than about 1%, 0.9%, 0.8%, 0.7%, 0.6%, 0.5%, 0.4%, 0.3%, 0.2% or 0.1% serum.

The terms “defined culture medium,” “defined medium,” and the like, as used herein, indicate that the identity and quantity of each medium ingredient is known. The term “defined,” when used in relation to a culture medium or a culture condition, refers to a culture medium or a culture condition in which the nature and amounts of approximately all the components are known. A culture, composition, or culture medium is “essentially free” of certain reagents, such as signaling inhibitors, animal components or feeder cells, when the culture, composition, and medium, respectively, have a level of these reagents lower than a detectable level using conventional detection methods known to a person of ordinary skill in the art or that these agents have not been extrinsically added to the culture, composition, or medium.

As used herein, “effective amount” means an amount of an agent sufficient to evoke a specified cellular effect according to the present invention.

The present invention has been described in terms of one or more preferred embodiments, and it should be appreciated that many equivalents, alternatives, variations, and modifications, aside from those expressly stated, are possible and within the scope of the invention.

### Example 1

The embodiment described here demonstrates the adaption of Drosophila adult muscle progenitor-like cells (DrAMPCs) to serum-free culture medium, transition of DrAMPCs from adherent to suspension culture, differentiation of DrAMPCs to multinucleated myotubes, and scaffold supported muscle growth from DrAMPC derived multinucleated myotubes.

Insect cells in particular are a promising platform for tissue engineering due to simple isolation and maintenance methods and existing infrastructure for industrial-scale production. Novel applications of tissue engineering include bioactuation and cultured meat. Dissimilar to medical applications, these goals are not constrained by concerns of immunogenicity, host-integration or in vivo-like function. Instead, design considerations for these technologies include large-scale, low-cost production in order for tissue-engineered commodities to be competitive against their conventional counterparts (e.g., electrical actuators, farmed meat). Bioactuators should also generate large contractile force, operate under a range of environmental conditions and incorporate control systems. In contrast, muscle produced for food applications does not need to function mechanically, but instead should be visually identical to farmed meat, palatable to consumers and provide nutritional benefit. Insect cells are potentially better suited than mammalian cells to fulfill many of these objectives.

Advances in tissue engineering have driven the emergence of new products and industries beyond the realm of regenerative medicine, including organs-on-a-chip, soft robotics and biofabricated food and materials. Specifically, skeletal muscle tissue engineering is now being applied for the development of muscle-powered bio-bots and bioengineered meat, also known as cultured meat^{1,2}. These applications are not constrained by concerns of immunogenicity, host-integration or in vivo-like function. Instead, relevant challenges include lowering costs and achieving efficient, large-scale production so that tissue engineered commodities can be competitive against their conventional counterparts (e.g., electrical actuators and agriculturally farmed meat)³. Muscle tissue produced as food should also be visually and texturally similar to farmed meat, appealing to consumers and nutritionally advantageous⁴. Conversely, bioactuators should generate large contractile force, operate under a range of environmental conditions and incorporate control systems⁵. Insect cells are potentially better suited than mammalian cells to address many of these objectives.

Commonly used cells for skeletal muscle tissue research include the mouse myoblast cell line C2C12, the rat myoblast cell line L6, and human cells obtained from primary lines or induced pluripotent stem cells⁶. These cell types are typically grown as adherent cultures at 37° C. with 5% carbon dioxide in sodium bicarbonate-buffered basal medium supplemented with fetal bovine serum. These conditions, while feasible for bench-scale culture, create hurdles for achieving cost-efficient production at scale for commercial cell-based products. Specifically, animal serum is costly and inconsistent, above ambient incubation temperatures require increased energy use, and adherent cell lines need complex substrates (e.g., microcarriers, hollow fibers) for high density growth in bioreactor systems^{7,8}.

In contrast, many insect cell lines are able to transition between adherent and suspension culture, and are best suited for temperatures within the ambient range of 19-30° C. and slightly acidic pH levels (6.2-6.4)⁹⁻¹¹. Unlike vertebrate cells, insect cells can be grown in a non-humidified environment and do not require CO₂ exchange¹². It is also reported to be relatively simple to adapt insect cells to both serum-free medium and suspension culture¹³. Furthermore, immortal or continuous insect cell lines are straightforward to obtain compared to vertebrate species, and many lines have been observed to retain their phenotype after over 120 cell doublings^{10,11}. In our prior study of Manduca sexta, primary cells maintained viability in culture for a month without media refreshment¹⁴. This set of unique culture characteristics make insect cells a particularly promising platform for novel applications of tissue engineering, and could contribute to scalable, cost-effective production systems for bioactuators and food.

Bioactuators are theorized to be advantageous over conventional actuators due to their efficiency and sustainability, and their capacity for self-assembly, self-healing and biodegradation¹⁵. The majority of bioactuator research at cellular and tissue levels has focused on mammalian cardiac or skeletal cells or insect dorsal vessel explants⁵. The general research strategy is to couple cultured cells or tissues with scaffold systems to perform mechanical work. For example, cardiomyocyte cell sheets have been combined with polydimethylsiloxane (PDMS) constructs to create a micropump with linear flow rates of 2 nL/mL¹⁶. PDMS molds have similarly been used with skeletal muscle cells to create microtissues under optogenetic control¹⁷. Insect tissue explants have also been of interest due to their tolerance of temperature fluctuations. Explanted DVT has been attached to PDMS molded devices which were operable at room temperature and generated 20 μN force¹⁸. In our own studies, we used cells isolated from embryos of the tobacco hawkmoth (Manduca sexta) to make simple muscle fiber bioactuators that contracted under a wide range of temperature, pH and nutrient conditions¹².

Cultured meat is another innovation derived from advances in muscle tissue engineering. By producing meat from cell cultures rather than whole organisms (e.g., farm animals), it is emerging as a potential solution to global food issues. Cultured muscle tissue production generally consists of (1) obtaining cells from an immortalized line or biopsy isolation, (2) proliferating the cells at scale in a serum-free media, and (3) differentiating the cells into muscle on an edible, degradable or reusable scaffold². Research in this field has gained traction since 2013, when a cultured beef burger “proof of concept” was produced⁴. Skeletal muscle development from porcine induced pluripotent stem cells has since been reported, offering a potentially reliable farm animal-derived cell line for food technology¹⁹. Primary muscle cell lines from chicken, cow, pig and horse have also been isolated and differentiated in vitro²⁰. Additional progress has been made on the development of sustainable and edible scaffold systems. Cellulose-based scaffolds fabricated from decellularized plants (e.g., spinach, apples) have been shown to support mammalian muscle growth^{21,22}.

Reliable cell sources, serum-free media formulations and 3D scaffold systems must be developed in order for insect muscle tissue engineering to be applied for bioactuator and cultured meat development. To date, in vitro insect muscle research has utilized explants or primary cells isolated from insect tissue. However, primary cell isolates result in mixed cell cultures and require frequent and labor-intensive isolation procedures¹⁴. A Drosophila melanogaster adult muscle immortalized progenitor-like cell line may be a promising initial cell source for insect tissue engineering because the cells (1) express GFP for ease of imaging, (2) are highly proliferative, (3) can differentiate upon treatment with the insect molting hormone 20-hydroxyecdysone¹⁰. It is important for the culture medium to be serum-free, low-cost and support muscle growth and differentiation. Fortunately, there are many commercial and “homemade” serum-free media formulations available for insect cells which can be verified or adapted to support muscle-specific cells²³. There is also a need for the design of affordable scaffold systems capable of supporting 3D insect muscle constructs. Mushroom-derived chitosan is a promising biomaterial for development of such scaffolds, as it is easily accessible, edible, widely used in tissue engineering and already incorporated in food products as an additive or dietary supplement²⁴. The combination of stable insect muscle cell lines, optimized media and scaffolding techniques will allow for further evaluation and analysis of the potential applications of insect muscle tissue engineering.

The embodiments described herein evaluate the potential of D. melanogaster muscle cells to serve as a platform for tissue engineering applications. We assessed moderate-scale production (e.g., serum-free culture, suspension culture, hormone regulation schemes), analyzed 3D culture systems via mushroom-derived chitosan scaffolding and quantified cellular levels of protein and minerals for nutritional insight.

### Results

Adaptation of Insect Muscle Cells to Serum Free Media and Inducing Single-Cell Suspension Culture—

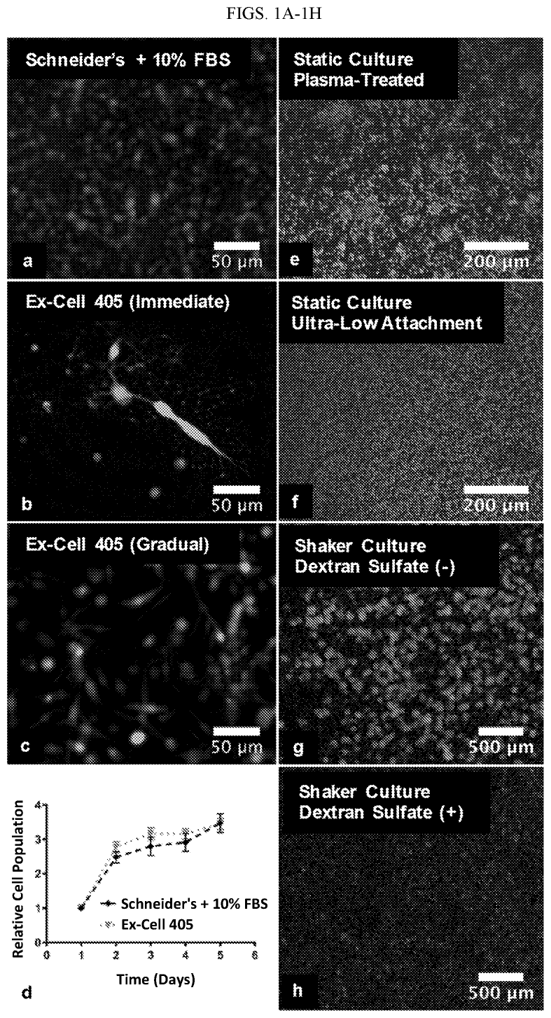

A primary goal of our research was to adapt insect muscle cells to serum-free media. We adapted the cells either immediately from 0% to 100% EC405 (FIG. 1B), or gradually over the course of two weeks by passaging the cells in increasing concentrations of EC405 (FIG. 1C). A subset of cells was maintained in serum-supplemented media as a control (FIG. 1A). Details of the adaptation schedule are listed in Table 1. The immediately adapted cells initially proliferated at rates equivalent to the control cells, however, after the first 48 hours the growth rate decreased. After one week in culture, the growth of immediately adapted cells was stagnant and the cell morphology appeared more neuron-like than myoblast-like; with multiple cell extensions protruding from the cell body (FIG. 1B). The gradually adapted cells retained their myoblast-like morphology identified by slight elongation (FIG. 1C) and exhibited comparable growth rates to controls (FIG. 1D). As shown in FIG. 1D, the insect muscle cells appear to exhibit diauxic growth. The two growth phases are distinguished by adherent growth and suspension growth. At low to medium cell densities, the cells are adherent. They proliferate until the surface is over-confluent (growth phase 1) and subsequently begin growing in suspension (growth phase 2).

TABLE 1
Adaptation of DrAMPCs to serum-free medium
Immediate Adaptation

| Pre-Adaptation | Day 1 | Day 2 | Day 3 | Day 4 | Day 5 |
|---|---|---|---|---|---|
| 100% Control | 100% EC405 | 100% EC405 | 100% EC405 | 100% EC405 | 100% EC405 |
| Media | | | | | Imaging |
| | | | | | (FIG. 1C) |

Gradual Adaptation

| Pre-Adaptation | Day 1-5 | Day 5-10 | Day 10-15 | Day 15-20 | Day 21 |
|---|---|---|---|---|---|
| 100% Control | 25% EC405 + | 50% EC405 + | 75% EC405 + | 100% EC405 | 100% EC405 |
| Media | 75% Control | 50% Control | 25% Control | | Imaging |
| | Media | Media | Media | | (FIG. 1C) |

Once the DrAMPC culture exhibited steady growth in EC405, the monolayer culture was transitioned to suspension culture. It was noted that when static culture flasks became over-confluent, the cells continued to grow in three-dimensional aggregates or in suspension. When the plasma-treated culture surface (FIG. 1E) was switched with an ultra-low attachment surface (FIG. 1F), the DrAMPCs did not attach and instead proliferated in suspension, at first as single cells and then forming aggregates, reaching a maximum cell density of 1E6 cells/mL after 5 days. The cells were then transitioned from static suspension culture to agitated suspension culture with the use of shaker flasks incubated at room temperature. In agitated suspension, the cells formed aggregates (FIG. 1G). However, the addition of 100 μg/mL dextran sulfate was sufficient to reduce aggregation and promote a single cell suspension (FIG. 1H). After expansion in suspension culture and removal of dextran sulfate, the cells transitioned back to adherent monolayers and retained a myoblast-like morphology.

Comparison of Mammalian Vs. Insect Muscle Cell Survival and Growth in Starvation Conditions—

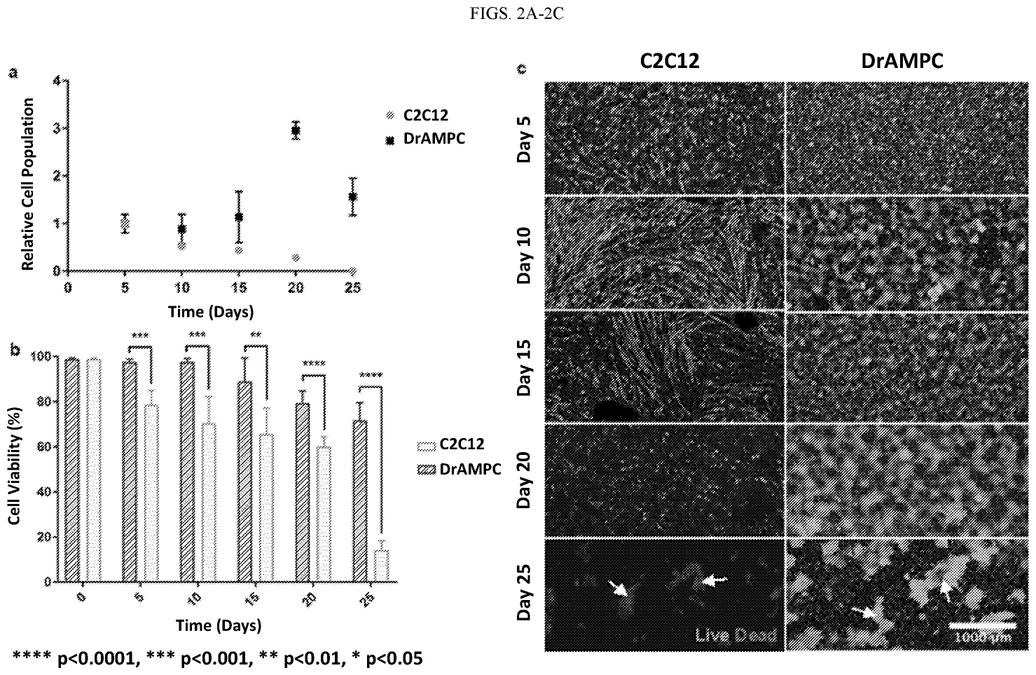

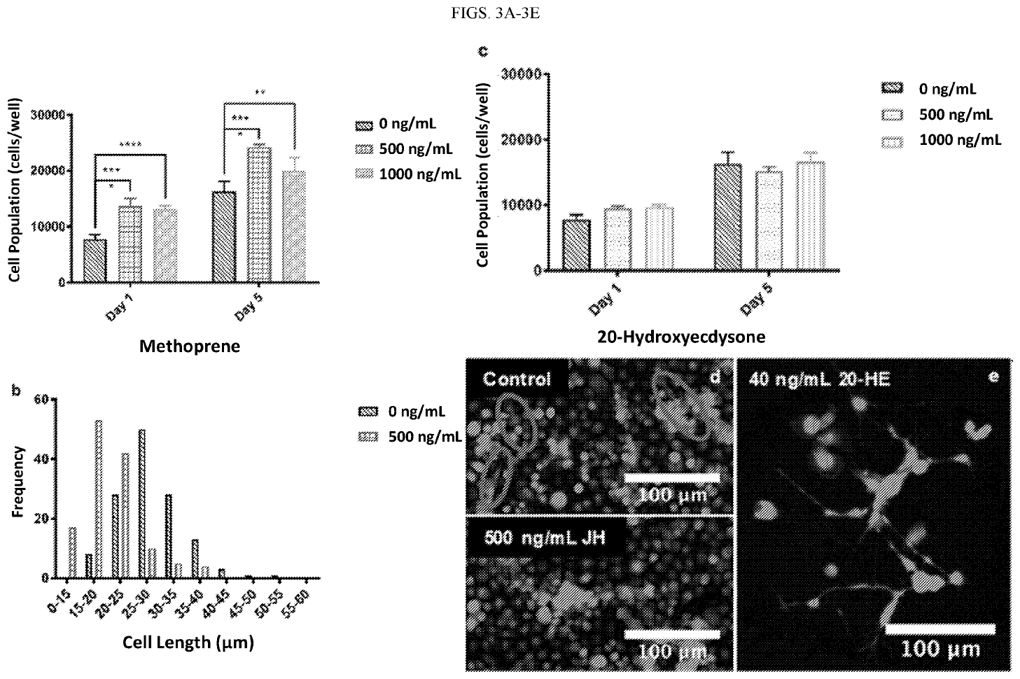

DrAMPC cells were noted to be capable of long-term survival and growth without media refreshment. To investigate this, DrAMPCs and C2C12 mouse myoblast cells were cultured in parallel to compare the survival of mammalian vs. insect muscle cells in limited nutrient conditions. Cells were initially fed 5 mL of media per well in a 6-well plate and subsequently left undisturbed until time point analysis. The C2C12 cells decreased in cell viability and total cell number over the course of 25 days in culture (FIGS. 2A and 2B). By day 25, the majority of cells had detached from the culture surface and were <20% viable. The DrAMPCs continued to proliferate up to day 20, after which most of the cells also detached from the cell surface. However, unlike the C2C12s, the detached DrAMPCs remained viable, with 70% viability observed after 25 days (FIGS. 2B and 2C). Only a small fraction of nonviable cells was observed in the center of large cell aggregates. In separate experiments, DrAMPCs were observed to survive for over two months without media refreshment. Viability only declined past 50% when components of the media began to crystalize as a result of water evaporation (FIG. 12).

Effect of Insect Hormones on Insect Muscle Cell Proliferation and Differentiation—

DrAMPCs were treated with methoprene (JH), a juvenile hormone mimic and insecticide, to investigate the effect of the hormone on cell proliferation. Cells treated with JH exhibited higher proliferation than control groups at both day 1 and 5 time points although the 500 ng/mL treatment level had a larger effect than the 1,000 ng/mL treatment (FIG. 3A). Aside from increasing cell numbers, JH treatment inhibited cell elongation, a preliminary sign of differentiation. The average length of JH-treated (500 ng/mL) cells was 8 μm less than the control cells (FIGS. 3B and 3D).

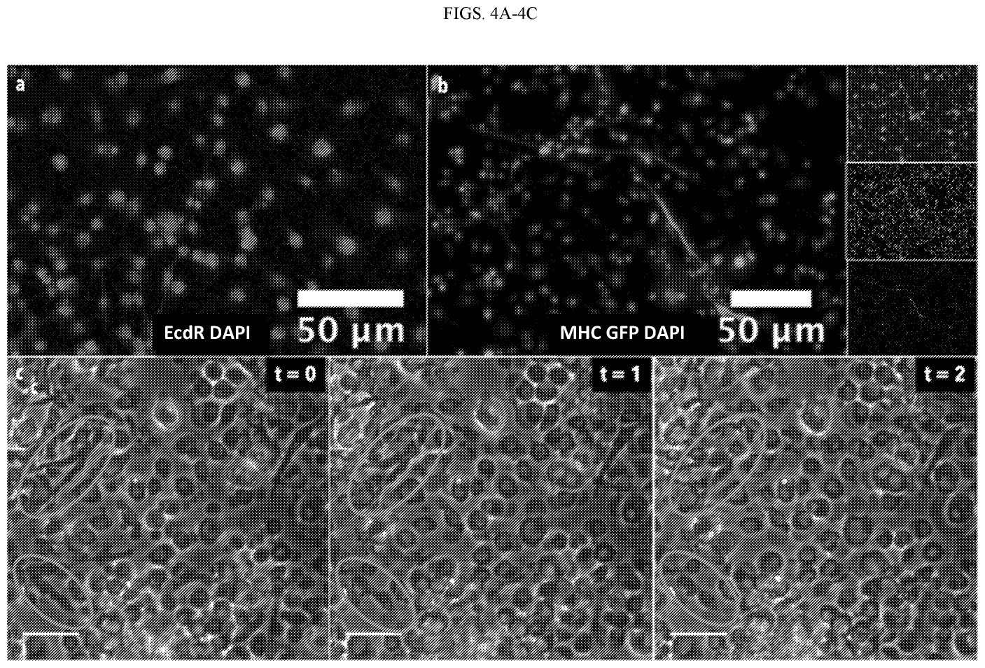

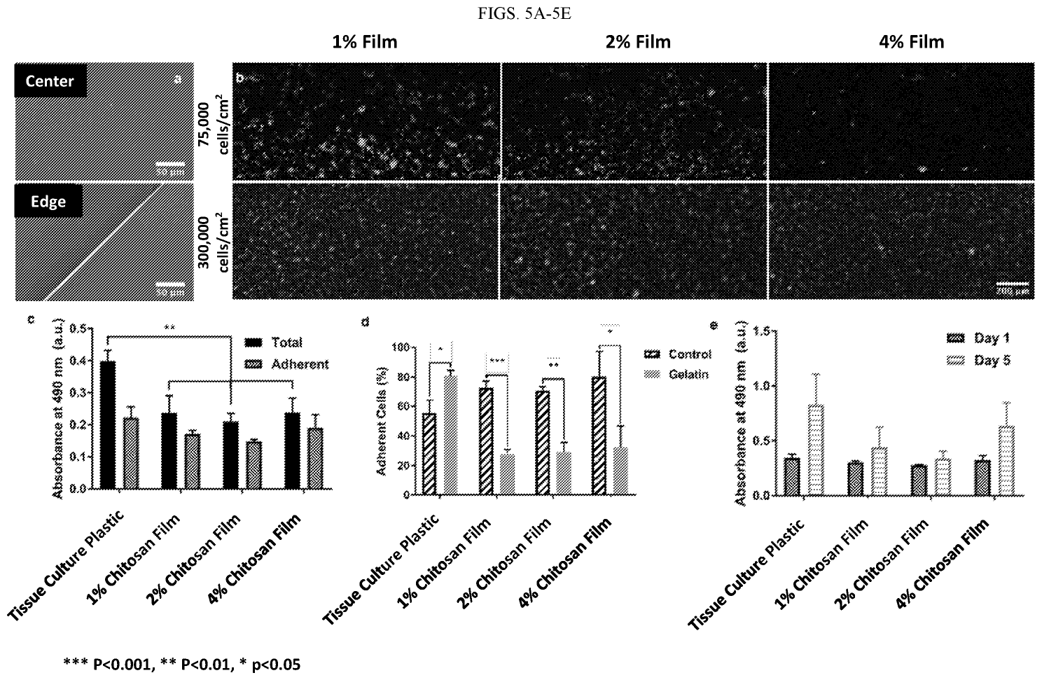

Cells were also treated with insect molting hormone, 20-hydroxyecdysone (20-HE). Growth rates of 20-HE treated cells did not significantly differ from control groups (FIG. 3C). Concentrations as low as 40 ng/mL 20-HE were able to trigger cell elongation, a preliminary sign of differentiation (FIG. 3E). When cells were treated with both JH and 20-HE, cell proliferation increased slightly but not significantly. It was noted that the cell population was consistently heterogeneous, with a fraction of cells elongating and fusing while the majority of cells remain spherical. To probe for differences in the cell population, we stained the cells for ecdysone receptor (EcdR), the receptor responsible for binding 20-HE to trigger molting in vivo or differentiation in vitro. All cells expressed EcdR, regardless of whether or not they initiated differentiation (FIG. 4A).

Induced Insect Muscle Cell Contraction by External Potassium—

The study that generated the DrAMPC line previously demonstrated that the cells cultured in serum-supplemented media undergo differentiation upon treatment with 20-HE. A small fraction of our EC405 adapted cells were also triggered by 20-HE to differentiate and form long, multinucleated myotubes that express myosin heavy chain; a skeletal muscle biomarker (FIG. 4B). Spontaneous cell contractions were not observed during our experiments, although they have been noted in previous research¹⁰. To demonstrate the contractile capacity of DrAMPCs, the cultures were treated with high concentrations of extracellular potassium (FIG. 4B). Intracellular potassium concentrations in Diptera muscle range between 142 and 179 mM²⁵. The cells did not contract when treated with 300 mM K⁺ but did contract when treated with 600 mM K⁺. The cells were 95% viable after potassium treatment. Cell fractional shortening was determined by images analysis. On average, the myoblasts (elongated cells) contracted by 31.6%±14.8% (n=12). In contrast, the spherical, proliferative precursor cells shortened by 1.6%±16.2% (n=14).

Insect Muscle Cell Growth on Chitosan-Based Scaffolds—

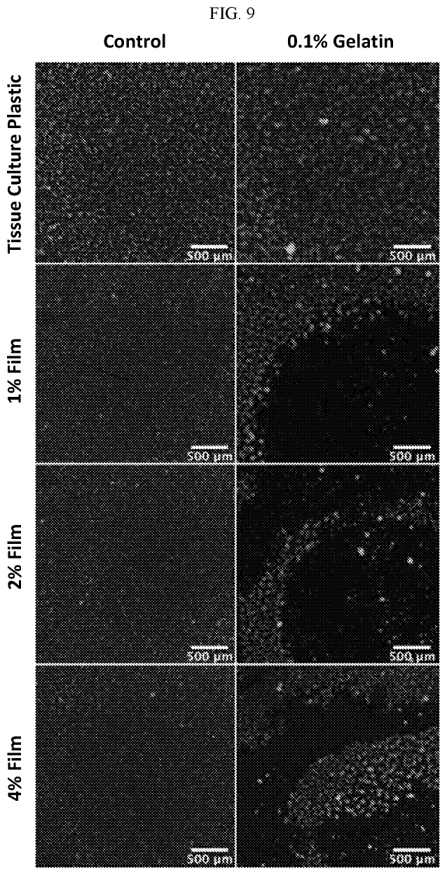

Flat films were fabricated from mushroom-based chitosan and seeded with DrAMPCs (FIG. 5A). After 24 hours and at medium seeding densities, DrAMPCs favored adhesion to low concentration films (1%, 2%), however, after multiple days in culture all films became confluent. At high seeding densities, cells attach to all films (FIG. 5B) after 24 hours. Cellular adhesion was assessed on tissue culture plastic controls and chitosan films. On day 1 in culture, the total cell population was compared to the adherent cell population by assaying wells as they were (Total) or after being aspirated and rinsed with PBS (Adherent) (FIG. 5C). The total cell population on tissue culture plastic was greater than the total cell population on the chitosan films, however, the differences in adherent cell populations between conditions were not statistically significant. This indicates that cells may grow more quickly on tissue culture plastic and the difference between total and adherent cells on the plastic may be due to cell growth in suspension after reaching confluence on the surface. To investigate the effect of a surface coating on cell adhesion, a subset of films was coated with 0.1% gelatin solution. Gelatin did improve cell adhesion to tissue culture plastic (FIG. 5D). On gelatin-coated films, cells appeared to grow in aggregates and were detached from the center of the well during the PBS rinse (FIG. 9). Because the detrimental effect was not observed in the control, it may be due to interactions (or lack thereof) between the chitosan surface and gelatin coating. At day 1, there were no significant differences between cell adherence to tissue culture plastic and the three film concentrations (FIG. 5E). By day 5, the tissue culture plastic conditions contained roughly twice the amount of adherent viable cells as day 1. Cells grew on the chitosan films to a lesser extent but more importantly remained adherent and viable.

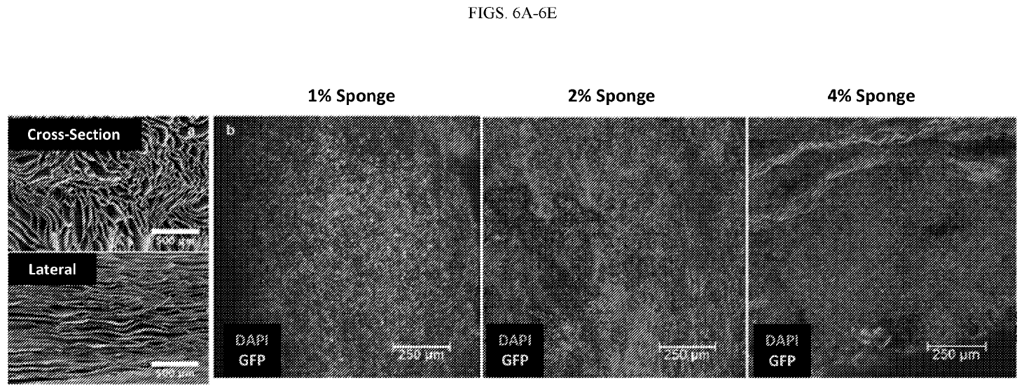

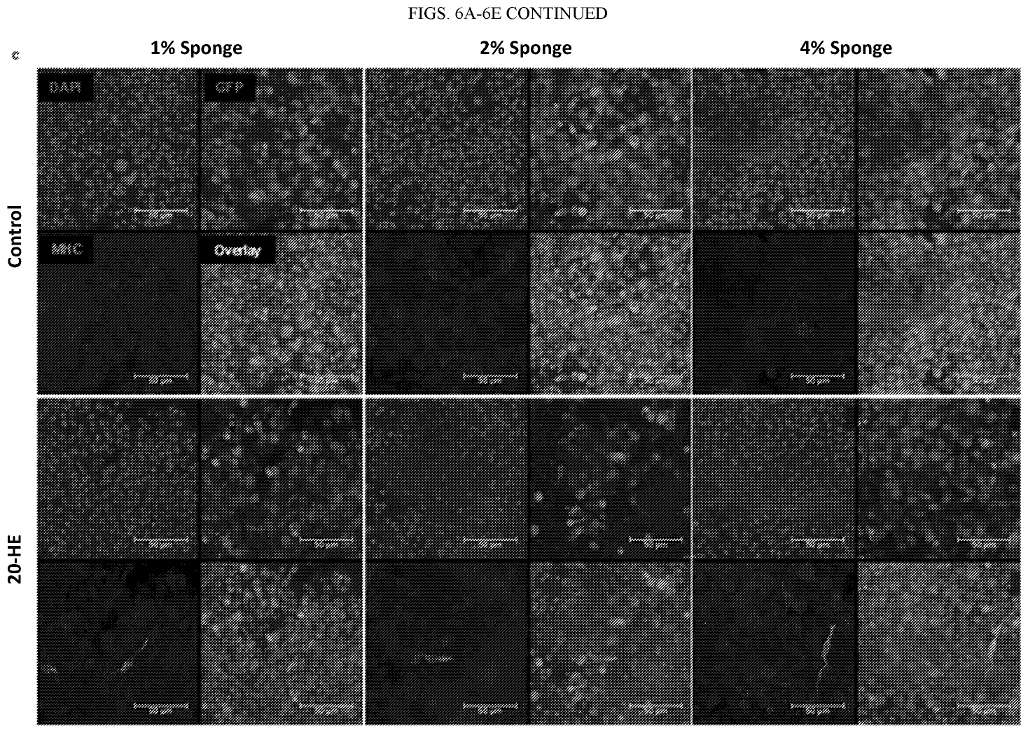

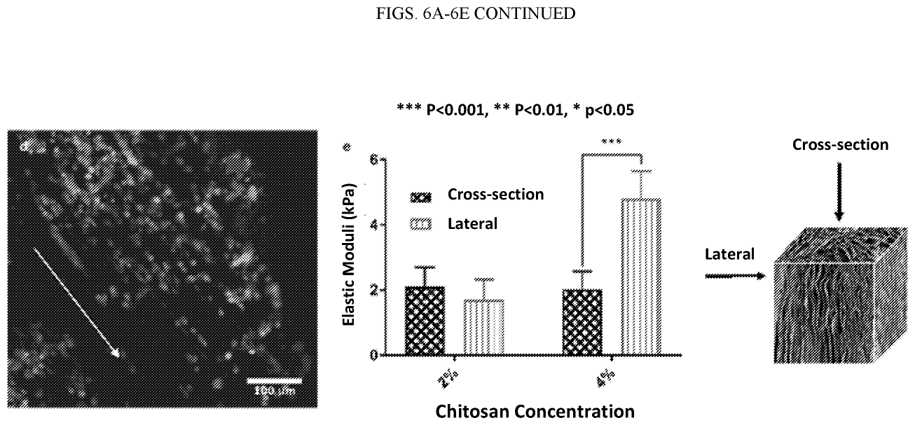

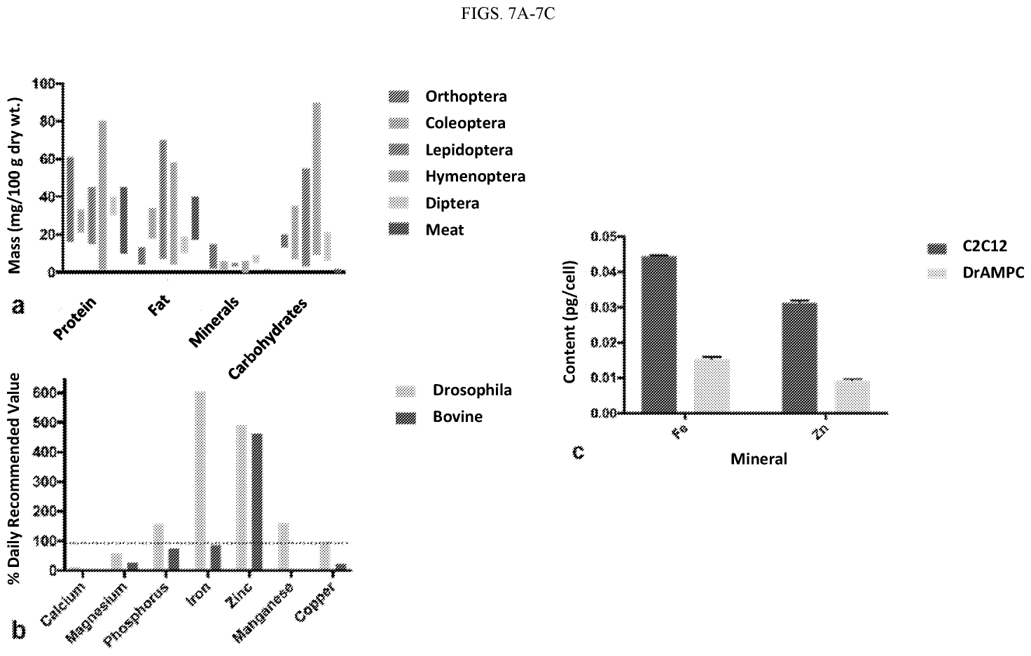

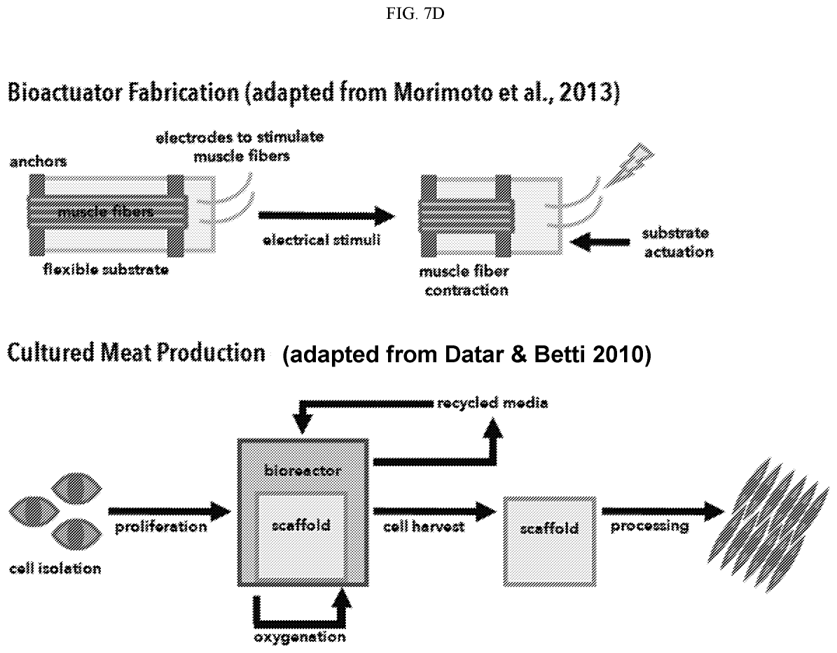

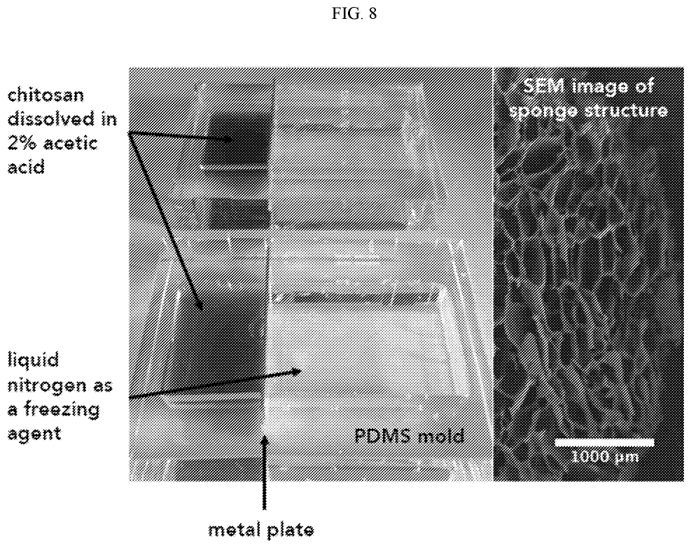

To translate the culture to 3D, chitosan sponges with aligned microtubular pores were fabricated using a directional freezing technique (FIG. 6A). DrAMPCs were cultured in chitosan sponges for one week (FIG. 6B) or two weeks (FIG. 11). At both time points, cells showed adhesion on all sponges (1, 2, 4% chitosan) and formed a nearly confluent monolayer. When treated with 20-hydroxyecdysone, a small fraction (<1%) of cells on the chitosan scaffolds differentiated (FIG. 6C). The average myocyte length on tissue culture plastic is 82 μm±40 μm (n=19) and the average myocyte length on chitosan is 68±34 μm (n=7). The lengths are not statistically significant via unpaired t-test with an p-value of 0.5568. The longest myocyte observed on plastic was 143 μm while the longest myocyte observed on chitosan was 120 μm. Myocyte lengths between chitosan scaffolds did not differ significantly. In select areas of growth, cells appeared to be in alignment with the length of the microtubular pores (FIG. 6D). Although cells grown on the 1% sponges exhibited strong adhesion, the sponges were extremely fragile and did not retain an aligned pore morphology when sectioned. After two weeks in culture, the 1% sponges disintegrated into sheet-like pieces while the 2% and 4% sponges were more durable. The mechanical properties of the sponges can be tuned via chitosan concentration²⁶. Elastic modulus values of the 2% and 4% sponges were determined via hydrated compression testing (FIG. 6E). The stiffness of both sponges in the cross-section direction was approximately 2 kPa. The moduli differed in the lateral direction, the 4% sponge being approximately twice as stiff as the 2% sponge. The mushroom-derived sponges in this study were less stiff than the comparable chitosan-derived sponges tested in Jana et al., 2013 (5 kPa versus 21 kPa for 4% sponges), a discrepancy which may be due to differences in initial molecular weight of the polymers²⁶. Both works show a similar trend of significant differences in moduli measured in the lateral direction rather than the cross-section direction of compression.

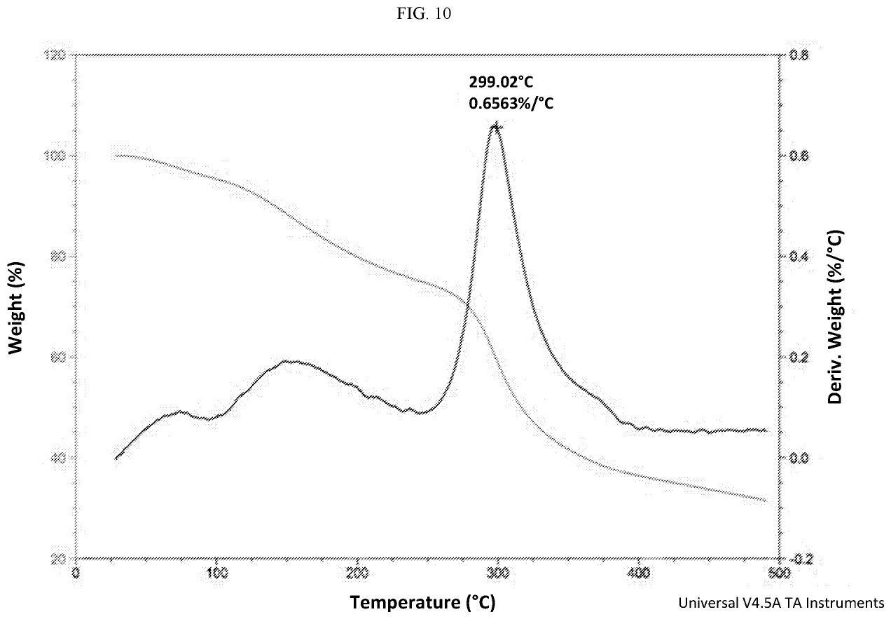

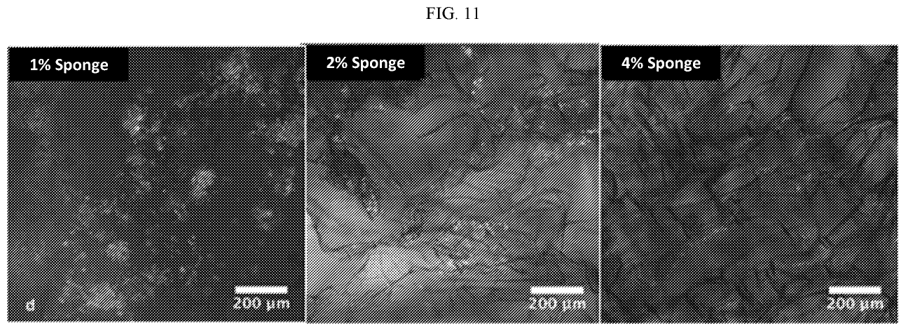

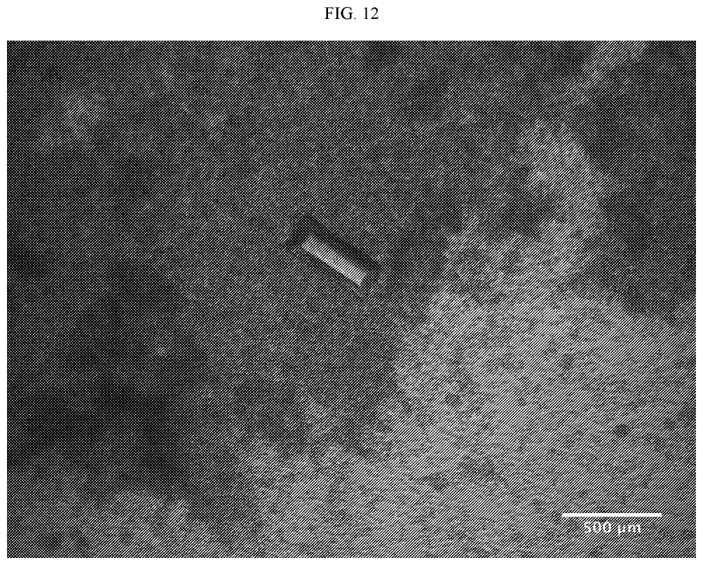

Preliminary data on the thermal degradation of chitosan was collected to provide initial insight into how chitosan sponges degrade under simulated cooking. Thermogravimetric analysis with ramped heat was performed from room temperature to 500° C. with a rate of 20° C. per minute. Chitosan powder begins to degrade at 325° C. and degradation is not significantly affected by heating rate (FIG. 10). There are three phases of degradation: (1) volatilization of residual materials, (2) chitosan chain degradation and (3) decomposition of remaining carbon²⁷.

Nutritional Properties of Insect Orders Compared to Vertebrate Muscle—

In order to probe nutrition differences between mammalian and insect muscle cells, we measured cellular protein and select mineral content of DrAMPC and C2C12 cells. Both cell cultures were assayed for total protein, iron and zinc (Table 2). Per cell, the C2C12 cells contain more (by factors of 2.5, 2.9 and 3.4, respectively) protein, iron and zinc than DrAMPC cells. However, the C2C12 cells (average diameter=19.60 μm) are much larger than DrAMPCs (average diameter=5.72 μm). Correcting for cell size, DrAMPCs contain a higher density of all three compounds per unit volume. These results correlate with in vivo nutrient values for the corresponding whole organisms as fruit fly tissue contains equivalent protein and a higher density of iron and zinc compared to mouse tissue (Table 3). This provides preliminary evidence that as insects are often more nutrient rich compared to mammalian tissue, insect cells may also be more nutritious than mammalian cells.

TABLE 2
Protein and mineral content, cell dimensions of
immortalized mouse and fly muscle cell lines.

| | | | | Suspended |
|---|---|---|---|---|
| | | | | cell |
| | Protein | Iron | Zinc | diameter |
| Cell Line | (pg/cell) | (pg/cell) | (pg/cell) | (μm) |
| C2C12 | 1393 ± 119 | 35.39 ± 0.34 | 156.14 ± 3.08 | 19.60 ± 1.95 |
| DrAMPC | 558 ± 67 | 12.09 ± 0.40 | 46.02 ± 2.16 | 5.72 ± 0.81 |

TABLE 3
Nutritional Comparison

| Organism | Protein (g/100 g) | Iron (mg/100 g) | Zinc (mg/100 g) |
|---|---|---|---|
| Mouse | 55.8 | 13.79 | 6.75 |
| Fruit Fly | 56.25 | 45.42 | 14.7 |

In a simple attempt to increase iron content, we cultured DrAMPCs in media supplemented with iron-fortified serum (136 μM Fe) and compared the cellular iron content to DrAMPCs cultured with media supplemented with conventional serum (24 μM Fe). The iron-fortified DrAMPCs contained twice the concentration of iron compared to the control, although the fortified media contained roughly 6-fold the amount of iron as the control media (Table 4). This indicates that while media can be formulated to influence nutritional content, there is a limit to the amount of minerals the cells can uptake.

TABLE 4
Iron content of bovine serum and insect muscle cells cultured
in media supplemented with 10% serum for five days.

| | Iron Content of | Iron Content of DrAMPCs |
|---|---|---|
| | Serum (μM) | Cultured in 10% Serum (μM) |
| Fetal Bovine | 24.35 ± 0.58 | 27.06 ± 0.90 |
| Serum | | |
| Iron-Fortified | 135.65 ± 5.51 | 62.40 ± 1.25 |
| Serum | | |

### Materials & Methods

Cell Culture—

Drosophila melanogaster adult muscle progenitor-like cells (DrAMPCs) were acquired from Kerafast (Boston, Mass.) (#EF4006). The cell line was originally immortalized by the Persimmon Research Group at Harvard University, and was derived from primary embryo cultures in which Gal4 drives Ras^{V12} and GFP expression. DrAMPCs were cultured in insect growth media composed of Schneider's Insect Medium from Sigma-Aldrich (St. Louis, Mo.) (#S0146) supplemented with 10% heat inactivated fetal bovine serum from ThermoFisher (Waltham, Mass.) (#16140) and 1% penicillin/streptomycin (ThermoFisher, #15140122). For serum-free growth media experiments, media consisted of Ex-Cell 405 Serum-Free Medium (Sigma-Aldrich, #14405C) and 1% penicillin/streptomycin. For static culture, DrAMPCs were cultured in either plasma-treated flasks (ThermoFisher, #156499) or ultra-low attachment flasks (Corning, N.Y.) (#CLS3814) and incubated at 19° C. in a temperature-controlled incubator from VWR (Radnor, Pa.) (#89511-416). For suspension culture, DrAMPCs were cultured in shaker flasks (ThermoFisher, #4115-0250) on an orbital shaker set at 40 rpm at room temperature. When indicated, dextran sulfate (Sigma-Aldrich, #67578) was supplemented to serum-free media by dissolution and sterile filtration with 0.2 μm bottle top filters (ThermoFisher, #595-3320). For hormone treatment and differentiation experiments, DrAMPCs were cultured in insect growth media or serum-free media supplemented with methoprene (Sigma-Aldrich, #33375) and/or 20-hydroxyecdysone (Sigma-Aldrich, #H5142) which were dissolved in DMSO (Sigma-Aldrich, #D8418). DrAMPCs were seeded at 75,000 or 300,000 cells/cm² in 2D culture and 1,000,000 cells/sponge in 3D culture. For mammalian controls, C2C12 cells from ATTC (Manassas, Va.) (#CRL-1772) were cultured in growth media composed of DMEM+Glutamax (ThermoFisher, #10566) supplemented with 10% heat inactivated fetal bovine serum and 1% penicillin/streptomycin. C2C12s were seeded at 10,000 cells/cm².

Adhesion, Proliferation and Viability Assays

Cell adhesion to tissue culture plastic and chitosan films was quantified via a MTS Assay from Promega (Madison, Wis.) (#G3582) by measuring absorbance after 2.5-hour incubation with the MTS reagent. Cell proliferation and viability were measured by fluorescence, Live/Dead kit (ThermoFisher, #L3224) stained image analysis or CyQuant proliferation assays (ThermoFisher, #C7026). For fluorescence measurements, GFP-expressing DrAMPCs were imaged on a fluorescence microscope over the course of a week using the automated multi-point capture feature. Fluorescence was quantified on Fiji software and normalized to the values determined at the first time-point. For Live/Dead stained image analysis, cells were stained following the kit protocols and imaged on a fluorescence microscope. The images were analyzed with Fiji to quantify the viability and total cell population over time. For CyQuant proliferation assays, cells were plated in 96-well plates for each time-point. At each time-point, media was blotted from the plates and plates were stored at −80° C. After all time-points were collected, plates were thawed to room temperature and stained with CyQuant working solution for 5 minutes. Microplate measurements were performed on a SpectraMax M2 reader from Molecular Devices (San Jose, Calif.). Cell populations were determined from a standard curve.

Staining and Imaging—

Cell viability was determined by a Live/Dead staining kit (ThermoFisher, #L3224). At time of assay, media was gently aspirated from the cell surface and cells were rinsed with phosphate buffered saline (PBS) (ThermoFisher, #14040133). Cells were stained with 2 μM calcein AM and 4 μM EthD-1 solution for 30 minutes at room temperature in the dark and imaged with a fluorescence microscope. For immunocytochemical staining, media was gently aspirated from cells and the cell surface was rinsed with PBS. Cells were fixed with 4% paraformaldehyde from Fisher Scientific (Hampton, N.H.) (#J61899AK) for 10 minutes and rinsed with PBS. Cells were then permeabilized with 0.5% Triton X-100 and rinsed with blocking buffer consisting of phosphate buffered saline, 5% fetal bovine serum and 0.05% sodium azide. Cells were incubated in blocking buffer for 15 minutes before addition of primary antibody, then incubated at 4° C. overnight. Cells were rinsed and incubated with fresh blocking buffer for 15 minutes before addition of secondary antibody, then incubated in the dark on ice for 1 hour. Again, cells were rinsed and incubated with fresh blocking buffer for 15 minutes before counterstaining and mounting with DAPI mounting medium from Abcam (Cambridge, UK) (#ab104139). Detailed antibody information is available in Table 5. Fluorescence imaging was performed on a Keyence microscope (Osaka, Japan) (#BZ-X700). Confocal imaging was performed on a TCS SP8 microscope from Leica Microsystems (Wetzlar, Germany).

TABLE 5
Immunocytochemistry Antibodies

| Figure | Primary Antibody | Secondary Antibody |
|---|---|---|
| 4A | Mouse Anti-EcR Antibody | Goat Anti-Mouse IgG Alexa |
| | (DSHB, #15C3) | Fluor ® 647 (Abcam, #ab150115) |
| | 1:50 Dilution | 1:200 Dilution |
| 5B | Rat Anti-Myosin Antibody | Donkey Anti-Rat IgG Alexa |
| | (Abcam, #ab51098) | Fluor ® 647 (Abcam, #ab150155) |
| | 1:500 Dilution | 1:200 Dilution |

Potassium Induced Contraction—

Cells were differentiated for 5 days before the experiment. High K+ concentration media was produced by dissolving KCl in growth media at the indicated concentration. Cells were imaged on a Phase Contrast microscope before growth media was aspirated and replaced with 600 mM K+ media. Cells were imaged over a duration of 15 minutes.

Scaffold Fabrication—

Mushroom chitosan of 100 kDa molecular weight from Chinova Bioworks (New Brunswick, Canada) was dissolved in 2% acetic acid in distilled water for 12 hours at room temperature on a stir plate. The chitosan solution was centrifuged at 16,880×g for 3 hours to remove undissolved particles and diluted in distilled water to the desired concentrations (1%, 2% and 4%). Concentrations were verified by drying small volumes at 60° C. for 2 hours and calculating dry weight/wet weight. To prepare films, the solution was cast on plastic and allowed to dry overnight. To prepare sponges, the solution was poured into PDMS molds with an aluminum sheet separating one side of the mold from a separate chamber. Liquid nitrogen was poured into the chamber opposite the chitosan solution and continually replenished until the entire solution had frozen across the temperature gradient. Samples were then lyophilized for 48 hours. Prior to sterilization and seeding, films and sponges were submerged in 1M sodium carbonate for 1 hour at room temperature and subsequently rinsed three times in distilled water for 20 minutes and soaked in distilled water overnight. For hydrated mechanical analysis, sectioned sponges were soaked in PBS prior to testing.

Cell Seeding—

Sponges used for seeding were cylinders 6 mm in diameter and 1.5 mm thick. They were sterilized for 24 hours with 70% ethanol and UV exposure, then soaked in growth media overnight. They were then seeded with 1,000,000 DrAMPCs in 50 μL of media and incubated for 4 hours before 1 mL of media was added to each well. Films were cast in 24-well plates and seeded at medium (75,000 DrAMPCs/cm²) or high (300,000 DrAMPCs/cm²). Media changes were performed once per week.

Mechanical Testing—

Compression tests were performed on an Instron 3366 from TA Instruments (New Castle, TE) with a strain rate of 1 mm/min to a total strain of 30%, and modulus values were calculated for the 2%-10% compression interval. Samples were tested in the hydrated state (immersed in 1×PBS). Sponge dimensions used were 8×8×8 mm cubes.

Nutritional Analysis—

DrAMPC and C2C12 cells were cultured and harvested into aliquots of 20 million and 10 million cells respectively for nutritional analysis. Cells were lysed with RIPA buffer (ThermoFisher, #89900) and 1% Halt Protease Inhibitor Cocktail EDTA-Free (ThermoFisher, #78425). Protein was quantified by a Pierce BCA Protein Assay Kit (ThermoFisher, #23225) according to manufacturer instructions. Iron and zinc were quantified by an Iron Assay Kit (Abcam, #ab83366) and Zinc Assay Kit (Abcam, #ab102507) according to manufacturer instructions. For iron fortification experiments, cells were cultured with 10% Iron-Fortified Bovine Serum (Sigma-Aldrich, #12138C). Microplate measurements were performed on a SpectraMax M2 reader (Molecular Devices).

Thermogravimetric Analysis—

Thermogravimetric analysis with ramped heat was performed on samples of chitosan sponge with a Thermogravimetric Analyzer (TA Instruments, #Q500). Samples were heated from room temperature to 500° C. at a rate of 20° C. per minute.

Statistical Analysis—

Statistical analysis was performed with GraphPad Prism 7.04 software. Error bars in column charts are standard deviations. Statistical significance was determined via two-way ANOVA and multiple comparisons with the Sidak post-hoc test or via multiple t tests with the Holm-Sidak post-hoc test with alpha=0.05. Additional info as well as the p-values for each statistically significant comparison are listed in Table 6.

TABLE 6
Statistical Analysis

| | Sample | | | | |
|---|---|---|---|---|---|
| Figure | Size | Replicates | Error Bars | Test | P-values |
| 1D | 5 | Separate wells on a 24- | Standard | NA | NA |
| | | well plates | deviations | | |
| 2A | 6 | Separate images (2 | Standard | NA | NA |
| | | images per well, 3 | deviations | | |
| | | wells on a 6-well | | | |
| | | plate) | | | |
| 2B | 6 | Separate images (2 | Standard | Multiple t tests with | Day 5 = 0.0001, |
| | | images per well, 3 | deviations | Holm-Sidak post-hoc | Day 10 = 0.0007, |
| | | wells on a 6-well | | test | Day 15 = 0.0081, |
| | | plate) | | | Day 20 = 0.0002, |
| | | | | | Day 25 <0.0001 |
| 3A | 5 | Separate wells on a 96- | Standard | Two-way ANOVA, | Day 1 (0 vs. 500 |
| | | well plate | deviations | multiple comparisons | ng/mL) < 0.0001, |
| | | | | with Sidak post-hoc | Day 1 (0 vs. 1000 |
| | | | | test | ng/mL) < 0.0001, |
| | | | | | Day 5 (0 vs. 500 |
| | | | | | ng/mL) < 0.0001, |
| | | | | | Day 5 (0 vs. 1000 |
| | | | | | ng/mL) = 0.0020 |
| 3C | 5 | Separate wells on a 96- | Standard | NA | NA |
| | | well plate | deviations | | |
| 5C | 3 | Separate wells on a 96- | Standard | NA | NA |
| | | well plate | deviations | | |
| 5D | 3 | Separate wells on a 96- | Standard | NA | NA |
| | | well plate | deviations | | |
| 6E | 5 | Separate samples | Standard | Multiple t tests with | NA |
| | | | deviations | Holm-Sidak post-hoc | |
| | | | | test | |

### DISCUSSION

Key challenges in the field of cultured muscle biomass for non-medical applications can be addressed with invertebrate cell sources. The main challenges include producing cells in a cost-efficient manner and transforming the cell mass into functional constructs. The majority of cell lines that have been scaled for industrial production are derived from humans (e.g., HEK 293, HeLa S3, WI-38), rodents (e.g., CHO-K1, NS0, BEK) and insects (e.g., S2, Sf9, High Five)²⁸. In most cases, these cells are not the end-product themselves, but rather are utilized to produce valuable therapeutic proteins (e.g., antibodies, cytokines, growth factors). The most commonly utilized cell lines share characteristics in that they are immortalized and can achieve high growth densities in serum-free, suspension culture.

So far, muscle cells have only been produced in relatively small quantities for research. A key step towards industrial-scale muscle cell culture is formulation of, and adaptation to, serum-free media. Serum is expensive and susceptible to batch-to-batch variability and cost fluctuations based on supply availability²⁹. For cultured meat applications, serum must be eliminated from culture media to reduce costs and allow for animal-free production. Serum-free media formulations have been reported for mammalian muscle cells, however, they are supplemented with animal-derived or recombinant proteins (e.g., fetuin, fibroblast growth factor, insulin)³⁻³³ and to our knowledge, not commercially available. In this work, a D. melanogaster muscle progenitor cell line was successfully adapted to Ex-Cell 405 serum-free insect media. Although this particular medium is proprietary, other insect cells have been adapted from Ex-Cell 405 media to defined, low-cost formulations with IPL-41 basal media (the preparation is publicly available)^{23,34}. Future work could include optimizing in-house media formulations.

The extended survival and proliferation of insect muscle cells in the absence of fresh media is demonstrated in this work and by previous studies from our lab¹⁴. We previously observed that primary cultures of M. sexta cells survived over 75 days in the same media and theorized the effect may be due to supporting yolk and fat cell types which store nutrients. The present work utilized an immortalized cell line and we observed cell survival without nutrient exchange for more than two months. We propose that the extended survival may be attributed to differences in mammalian versus insect metabolism. In a study comparing mammalian and insect glucose and glutamine metabolism, insect cells uniquely channeled glucose metabolites into the tricarboxylic acid cycle which produced more energy per unit glucose than glycolysis³⁵. Insect cells also do not produce significant lactate, the accumulation of which significantly lowers the pH of mammalian muscle cell cultures.

Mammalian muscle cells generally grow as adherent rather than suspension cultures. Cost-efficient production would require suspension cultures of stem cells with myogenic-lineage, adaptation of muscle progenitor cells to suspension cultures or development of more complex bioreactor systems suitable for adherent cell types (e.g., microcarriers, hollow fibers). The results from the present study demonstrated that the DrAMPC line of invertebrate muscle cells can be grown in single-cell suspension cultures and transition back to monolayer cultures after expansion. Dextran sulfate supplementation was sufficient to reduce cell aggregation, and shaker flask agitation kept cells in suspension. Removal of dextran sulfate and reduction of cell density in static cultures promoted monolayer culture and cell elongation. This versatility and adaptability allows for streamlined production and scale-up.

For industrial bioprocesses, it will be advantageous to control proliferation and differentiation of muscle cells via external factors. Methoprene and 20-hydroxyecdysone have previously been shown to affect the growth and development of insect cells in vitro³⁶⁻³⁸. In the present study, methoprene increased proliferation and 20-hydroxyecdysone triggered differentiation in both serum-supplemented and serum-free cultures. Methoprene is used as an insecticide generated via chemical synthesis' for the production of many agricultural products. It poses minimal risk to the environment, and guidelines for human consumption have been developed (recommended exposure is set at 0.3750 mg/day for the average adult)⁴⁰. The hormone 20-hydroxyecdysone is produced by plants as well as insects and has been produced in plant in vitro cultures⁴¹. It is also safe for human consumption and even promoted as a fitness supplement⁴².

When applying this technology for food purposes, it is important to consider potential hazards to human health. Human health risks could arise from the original insect species, the derived cell cultures or the circulated media. Although many insect species are edible and nutritious, some species present risks in the form of anti-nutrient substances, allergens, synthesized defense toxins, pesticides and microbial pathogens. These risks can generally be avoided by selecting safe species, decontamination and raising the insects on nontoxic feed⁴³. D. melanogaster flies are safe to eat and sold by companies to eat as whole larvae, oils, or powder. Because D. melanogaster are safe to consume, we assume cells cultured from fruit flies would be safe to consume as well, given that the cultures are fed with food-grade growth media. Human pathogens have not been detected in insect cell cultures, however, some cell lines can perform posttranslational modifications to proteins that may trigger human allergies¹¹. Due to ExCell 405 being a proprietary media formulation, we are unsure whether it contains any components that could be dangerous for consumption. However, in-house serum-free insect media formulations typically contain basal media, soy protein extract, yeast extract and lipids that are not toxic to humans²³. Cultured insect cells may be safer than whole insects as the restricted number of cell types and controlled environment may eliminate the presence of certain allergens and toxins. While primary cell lines are likely safe, concerns may rise for continuous or immortalized cell lines. Spontaneously immortalized cell lines may develop mutations overtime, and it is unclear whether this may pose a safety risk. Genetically immortalized cells may present additional concerns. The D. melanogaster adult muscle precursor-like cell line used in this study was immortalized via the oncogenic protein RasV12 and expresses GFP. Ingestion of GFP is unlikely to be detrimental to human health7. Farmed meat likely contains cancerous tissue at times and this is probably not a health risk as (1) cancer is not contagious and (2) proper cooking and digestion processes should breakdown cancerous cells. Similarly, dead cultured cells or tissues should not pose a risk for human consumption. Regardless, it is important for further research to take place surrounding the ingestion of cultured and genetically modified cell-based foods.

Large, aligned muscle tissues are necessary to produce bioactuators with useful contractile force and structured meat-like tissues for food. The construction of three-dimensional and functional mammalian muscle tissues has been widely pursued⁴⁴⁻⁴⁸. Insect muscle constructs are remarkably less researched and consist mainly of muscle explants or primary cultures self-assembled in PDMS molds^{12,49}. We evaluated mushroom-derived chitosan as a scaffolding biomaterial to support in vitro culture of insect muscle cells. Chitosan was selected as the biomaterial of choice for this system as it is (1) a well-researched biomaterial in the field of tissue engineering, (2) edible, (3) inexpensive, (4) can be acquired from animal-free sources (fungi, microbes), (5) is approved for human consumption, (6) may supply health benefits and (7) scaffold techniques for three-dimensional muscle culture have been established²⁶. In a feasible production scenario, insect cells would first be expanded in a single cell suspension culture and subsequently seeded on a static scaffold for tissue maturation. Therefore, adhesion, viability and differentiation on cell-scaffold constructs are more important factors than growth. Although gelatin coating did not increase cell adhesion to chitosan films, a different outcome may result from creating a chitosan-gelatin composite solution before casting the film as opposed to a gelatin coating. Other materials to investigate include collagen and laminin, although it is important to remember the design requirement of minimizing animal-derived byproducts. Because cells can form a confluent layer on non-functionalized chitosan films and sponges, a coating material may be unnecessary.

The purpose of modulating scaffold stiffness is to have some control over the texture and palatability of the end-product. One long-term goal of the research area is to have three-dimensional cell-scaffold constructs with mechanical properties similar to meat. The elastic modulus of bovine muscle (mixed fiber orientation) is ˜1.5 kPa. Chitosan sponges can be fabricated to have a similar stiffness, implying a chitosan-based cultured meat product could have similar texture to familiar meat products. It will also be important to investigate the mechanical properties of seeded scaffolds and the effect of cooking temperature and time.

The most prevalent challenge in this study was promoting homogenous differentiation, as the majority of cells retained a spherical morphology indicative of their proliferative state. However, on plastic and chitosan scaffolds, myosin heavy chain-expressing cells could be generated (albeit at low efficiency) after treatment with 20-hydroxyecdysone. In all control samples (plastic and sponges not treated with 20-hydroxyecdyone), no myosin heavy chain-expressing cells were observed. This supports the conclusion that while DrAMPCs have the capacity to differentiate to a muscle phenotype on plastic and 2D and 3D chitosan scaffolds, the system is lacking an element or elements necessary for robust differentiation.

### REFERENCES

(1) Ricotti, L.; Trimmer, B.; Feinberg, A. W.; Raman, R.; Parker, K. K.; Bashir, R.; Sitti, M.; Martel, S.; Dario, P.; Menciassi, A. Biohybrid Actuators for Robotics: A Review of Devices Actuated by Living Cells. Sci. Robot 2017, 2, 29, DOI: 10.1126/scirobotics.aaq0495.
(2) Datar, I.; Betti, M. Possibilities for an in Vitro Meat Production System. Innov. Food Sci. Emerg. Technol. 2010, 11 (1), 13-22, DOI: 10.1016/j.ifset.2009.10.007.
(3) Verbeke, W.; Sans, P.; Van Loo, E. J. Challenges and Prospects for Consumer Acceptance of Cultured Meat. J. Integr. Agric. 2015, 14 (2), 285-294, DOI: 10.1016/S2095-3119(14)60884-4.
(4) Post, M. J. Cultured Beef: Medical Technology to Produce Food. J. Sci. Food Agric. 2014, 94 (6), 1039-1041, DOI: 10.1002/jsfa.6474.
(5) Bashir, R.; Chan, V.; Asada, H. H. Utilization and Control of Bioactuators across Multiple Length Scales. 14, 653, DOI: 10.1039/c31c50989c.
(6) Klumpp, D.; Horch, R. E.; Kneser, U.; Beier, J. P. Engineering Skeletal Muscle Tissue—New Perspectives in Vitro and in Vivo. J. Cell. Mol. Med. 2010, 14 (11), 2622-2629, DOI: 10.1111/j.1582-4934.2010.01183.x.
(7) Verbruggen, S.; Luining, D.; van Essen, A.; Post, M. J. Bovine Myoblast Cell Production in a Microcarriers-Based System. Cytotechnology 2018, 70 (2), 503-512, DOI: 10.1007/s10616-017-0101-8.
(8) Zhang, Y.; Stobbe, P.; Silvander, C. O.; Chotteau, V. Very High Cell Density Perfusion of CHO Cells Anchored in a Non-Woven Matrix-Based Bioreactor. J. Biotechnol. 2015, 213, 28-41, DOI: 10.1016/j.jbiotec.2015.07.006.
(9) Beas-Catena, A.; Sánchez-Mirón, A.; Garcia-Camacho, F.; Molina-Grima, E. Adaptation of the Se301 Insect Cell Line to Suspension Culture. Effect of Turbulence on Growth and on Production of Nucleopolyhedrovius (SeMNPV). Cytotechnology 2011, 63 (6), 543-552, DOI: 10.1007/s10616-011-9387-0.
(10) Mary-Lee Dequéant, B.; Fagegaltier, D.; Hu, Y.; Spirohn, K.; Simcox, A.; Hannon, G. J. Discovery of Progenitor Cell Signatures by Time-Series Synexpression Analysis during Drosophila Embryonic Cell Immortalization. Proc Natl Acad Sci 2015, 42112 (10), 12974-12979, DOI: 10.1073/pnas.1517729112.
(11) van Oers, M. M.; Lynn, D. E. Insect Cell Culture. In Encyclopedia of Life Sciences; John Wiley & Sons, Ltd: Chichester, UK, 2010, DOI: 10.1002/9780470015902.a0002574.pub2.
(12) Baryshyan, A. L.; Domigan, L. J.; Hunt, B.; Trimmer, B. A.; Kaplan, D. A. Self-Assembled Insect Muscle Bioactuators with Long Term Function under a Range of Environmental Conditions. R. Soc. Chem. Adv. 2014, 75 (4), 39962-39968, DOI: 10.1039/C4RA08438A.
(13) Ikonomou, L.; Schneider, Y.-J.; Agathos, S. N. Insect Cell Culture for Industrial Production of Recombinant Proteins. Appl. Microbiol. Biotechnol. 2003, 62 (1), 1-20, DOI: 10.1007/s00253-003-1223-9.
(14) Baryshyan, A. L.; Woods, W.; Trimmer, B. A.; Kaplan, D. L. Isolation and Maintenance-Free Culture of Contractile Myotubes from Manduca sexta Embryos. PLoS One 2012, 7 (2), DOI: 10.1371/journal.pone.0031598.
(15) Raman, R.; Grant, L.; Seo, Y.; Cvetkovic, C.; Gapinske, M.; Palasz, A.; Dabbous, H.; Kong, H.; Pinera, P. P.; Bashir, R. Damage, Healing, and Remodeling in Optogenetic Skeletal Muscle Bioactuators. Adv. Healthc. Mater. 2017, 6 (12), 1-9, DOI: 10.1002/adhm.201700030.
(16) Tanaka, Y.; Morishima, K.; Shimizu, T.; Kikuchi, A.; Yamato, M.; Okano, T.; Kitamori, T. An Actuated Pump On-Chip Powered by Cultured Cardiomyocytes. Lab Chip 2006, 6 (3), 362, DOI: 10.1039/b515149j.
(17) Sakar, M. S.; Neal, D.; Boudou, T.; Borochin, M. A.; Li, Y.; Weiss, R.; Kamm, R. D.; Chen, C. S.; Asada, H. H. Formation and Optogenetic Control of Engineered 3D Skeletal Muscle Bioactuators. Lab Chip 2012, 12 (23), 4976-4985, DOI: 10.1039/c21c40338b.
(18) Akiyama, Y.; Odaira, K.; Iwabuchi, K.; Morishima, K. Long-Term and Room Temperature Operable Bio-Microrobot Powered by Insect Heart Tissue. Proc. IEEE Int. Conf. Micro Electro Mech. Syst. 2011, 145-148, DOI: 10.1109/MEMSYS.2011.5734382.
(19) Genovese, N. J.; Domeier, T. L.; Telugu, B. P. V. L.; Roberts, R. M. Enhanced Development of Skeletal Myotubes from Porcine Induced Pluripotent Stem Cells. Sci. Rep. 2017, 7 (August 2016), 41833, DOI: 10.1038/srep41833.
(20) Baquero-Perez, B.; Kuchipudi, S. V; Nelli, R. K.; Chang, K.-C. A Simplified but Robust Method for the Isolation of Avian and Mammalian Muscle Satellite Cells. BMC Cell Biol. 2012, 13 (1), 16, DOI: 10.1186/1471-2121-13-16.
(21) Modulevsky, D. J.; Lefebvre, C.; Haase, K.; Al-Rekabi, Z.; Pelling, A. E. Apple Derived Cellulose Scaffolds for 3D Mammalian Cell Culture. PLoS One 2014, 9 (5), e97835, DOI: 10.1371/journal.pone.0097835.
(22) Gershlak, J. R.; Hernandez, S.; Fontana, G.; Perreault, L. R.; Hansen, K. J.; Larson, S. A.; Binder, B. Y. K.; Dolivo, D. M.; Yang, T.; Dominko, T.; et al. Crossing Kingdoms: Using Decellularized Plants as Perfusable Tissue Engineering Scaffolds. Biomaterials 2017, 125, 13-22, DOI: 10.1016/J.BIOMATERIALS.2017.02.011.
(23) Donaldson, M. S.; Shuler, M. L. Low-Cost Serum-Free Medium for the BTI-Tn5B1-4 Insect Cell Line. Biotechnol. Prog. 1998, 14 (4), 573-579, DOI: 10.1021/bp9800541.
(24) Croisier, F.; Jerome, C. Chitosan-Based Biomaterials for Tissue Engineering. Eur. Polym. J. 2013, 49 (4), 780-792, DOI: 10.1016/J.EURPOLYMJ.2012.12.009.
(25) Djamgoz, M. B. A. Insect Muscle: Intracellular Ion Concentrations and Mechanisms of Resting Potential Generation. J. Insect Physiol. 1987, 33 (5), 287-314, DOI: 10.1016/0022-1910(87)90118-1.
(26) Jana, S.; Cooper, A.; Zhang, M. Chitosan Scaffolds with Unidirectional Microtubular Pores for Large Skeletal Myotube Generation. Adv. Healthc. Mater. 2013, 2 (4), 557-561, DOI: 10.1002/adhm.201200177.
(27) Hong, P.-Z.; Li, S.-D.; Ou, C.-Y.; Li, C.-P.; Yang, L.; Zhang, C.-H. Thermogravimetric Analysis of Chitosan. J Appl Polym Sci 2007, 105, 547-551, DOI: 10.1002/app.25920.
(28) Bleckwenn, N. A.; Shiloach, J. Large-Scale Cell Culture. In Current Protocols in Immunology; John Wiley & Sons, Inc.: Hoboken, N.J., USA, 2004, DOI: 10.1002/0471142735.ima01us59.
(29) Gstraunthaler, G. Alternatives to the Use of Fetal Bovine Serum: Serum-Free Cell Culture. ALTEX 2003, 20 (4), 275-281, DOI: 10.1.1.319.6024.
(30) Claycomb, W. C. Culture of Cardiac Muscle Cells in Serum-Free Media. Exp. Cell Res. 1981, 131 (1), 231-236, DOI: 10.1016/0014-4827(81)90423-7.
(31) Shiozuka, M.; Kimura, I. Improved Serum-Free Defined Medium for Proliferation and Differentiation of Chick Primary Myogenic Cells. 2000, 201-207, DOI: 10.2108/zsj.17.201.
(32) Florini, J. R.; Roberts, S. B. A Serum-Free Medium for the Growth of Muscle Cells in Culture. In Vitro 1979, 15 (12), 983-992, DOI: 10.1007/BF02619157.
(33) Allen, R. E.; Dodson, M. V.; Luiten, L. S.; Boxhorn, L. K. A Serum-Free Medium That Supports the Growth of Cultured Skeletal Muscle Satellite Cells. Vitr. Cell. Dev. Biol. 1985, 21 (11), 636-640, DOI: 10.1007/BF02623296.
(34) Weiss, S. A.; Smith, G. C.; Kalter, S. S.; Vaughn, J. L. Improved Method for the Production of Insect Cell Cultures in Large Volume. In Vitro 1981, 17 (6), 495-502, DOI: 10.1007/BF02633510.
(35) Neermann, J.; Wagner, R. Comparative Analysis of Glucose and Glutamine Metabolism in Transformed Mammalian Cell Lines, Insect and Primary Liver Cells. J. Cell. Physiol. 1996, 166 (1), 152-169, DOI: 10.1002/(SICI)1097-4652(199601)166:1<152::AID-JCP18>3.0.00; 2-H.
(36) Oberlander, H.; Leach, C. E.; Shaaya, E. Juvenile Hormone and Juvenile Hormone Mimics Inhibit Proliferation in a Lepidopteran Imaginal Disc Cell Line. J. Insect Physiol. 2000, 46 (3), 259-265, DOI: 10.1016/S0022-1910(99)00178-X.
(37) Giraudo, M.; Califano, J.; Hilliou, F.; Tran, T.; Taquet, N.; Feyereisen, R.; Le Goff, G. Effects of Hormone Agonists on Sf9 Cells, Proliferation and Cell Cycle Arrest. PLoS One 2011, 6 (10), e25708, DOI: 10.1371/journal.pone.0025708.
(38) Cherbas, L.; Koehler, M. M. D.; Cherbas, P. Effects of Juvenile Hormone on the Ecdysone Response of Drosophila Kc Cells. Dev. Genet. 1989, 10 (3), 177-188, DOI: 10.1002/dvg.1020100307.
(39) Odinokov, V. N.; Ishmuratov, G. Y.; Kharisov, R. Y.; Serebryakov, E. P.; Tolstikov, G. A. Synthesis OfS-(+)-Methoprene. Russ. Chem. Bull. 1993, 42 (1), 98-99, DOI: 10.1007/BF00699984.
(40) Lawler, S. P. Environmental Safety Review of Methoprene and Bacterially-Derived Pesticides Commonly Used for Sustained Mosquito Control. Ecotoxicol. Environ. Saf 2017, 139, 335-343, DOI: 10.1016/J.ECOENV.2016.12.038.
(41) Thiem, B.; Kikowska, M.; Maliński, M. P.; Kruszka, D.; Napierala, M.; Florek, E. Ecdysteroids: Production in Plant in Vitro Cultures. Phytochem. Rev. 2017, 16 (4), 603, DOI: 10.1007/S11101-016-9483-Z.
(42) Wilborn, C. D.; Taylor, L. W.; Campbell, B. I.; Kerksick, C.; Rasmussen, C. J.; Greenwood, M.; Kreider, R. B. Effects of Methoxyisoflavone, Ecdysterone, and Sulfo-Polysaccharide Supplementation on Training Adaptations in Resistance-Trained Males. J. Int. Soc. Sports Nutr. 2006, 3 (2), 19-27, DOI: 10.1186/1550-2783-3-2-19.
(43) Rumpold, B. A.; Schluter, 0. K. Nutritional Composition and Safety Aspects of Edible Insects. Mol. Nutr. Food Res. 2013, 57 (5), 802-823, DOI: 10.1002/mnfr.201200735.
(44) Patil, P.; Szymanski, J. M.; Feinberg, A. W. Defined Micropatterning of ECM Protein Adhesive Sites on Alginate Microfibers for Engineering Highly Anisotropic Muscle Cell Bundles. Adv. Mater. Technol. 2016, 1 (4), 1600003, DOI: 10.1002/admt.201600003.
(45) Shimizu, K.; Fujita, H.; Nagamori, E. Alignment of Skeletal Muscle Myoblasts and Myotubes Using Linear Micropatterned Surfaces Ground with Abrasives. Biotechnol. Bioeng. 2009, 103 (3), 631-638, DOI: 10.1002/bit.22268.
(46) Ostrovidov, S.; Hosseini, V.; Ahadian, S.; Fujie, T.; Parthiban, S. P.; Ramalingam, M.; Bae, H.; Kaji, H.; Khademhosseini, A. Skeletal Muscle Tissue Engineering: Methods to Form Skeletal Myotubes and Their Applications. Tissue Eng. Part B. Rev. 2014, 20 (5), 403-436, DOI: 10.1089/ten.TEB.2013.0534.
(47) Cittadella Vigodarzere, G.; Mantero, S. Skeletal Muscle Tissue Engineering: Strategies for Volumetric Constructs. Front Physiol 2014, 5 (September), 362, DOI: 10.3389/fphys.2014.00362.
(48) Chen, S.; Nakamoto, T.; Kawazoe, N.; Chen, G. Engineering Multi-Layered Skeletal Muscle Tissue by Using 3D Microgrooved Collagen Scaffolds. Biomaterials 2015, 73, 23-31, DOI: 10.1016/j.biomaterials.2015.09.010.
(49) Akiyama, Y.; Hoshino, T.; Iwabuchi, K.; Morishima, K. Room Temperature Operable Autonomously Moving Bio-Microrobot Powered by Insect Dorsal Vessel Tissue. PLoS One 2012, 7 (7), e38274, DOI: 10.1371/journal.pone.0038274.
(50) Luedeman, R.; Levine, R. B. Neurons and Ecdysteroids Promote the Proliferation of Myogenic Cells Cultured from the Developing Adult Legs of Manduca sexta. Dev. Biol. 1996, 173, 51-68, DOI: 10.1006/dbio.1996.0006.

### Example 2

The embodiment demonstrated in this example shows the culture of additional insect cells lines and additional culture conditions suitable for use in the compositions and methods described herein.

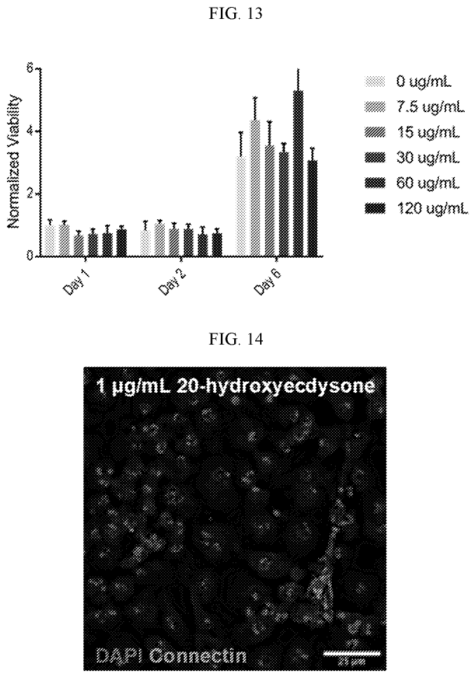

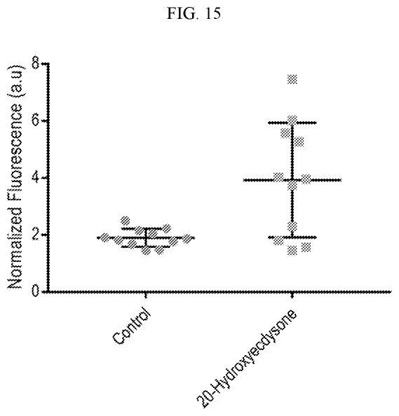

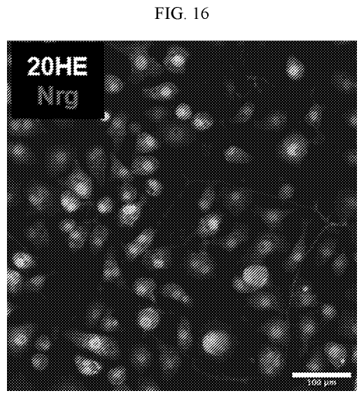

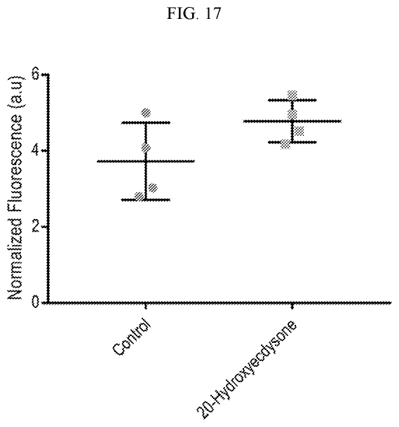

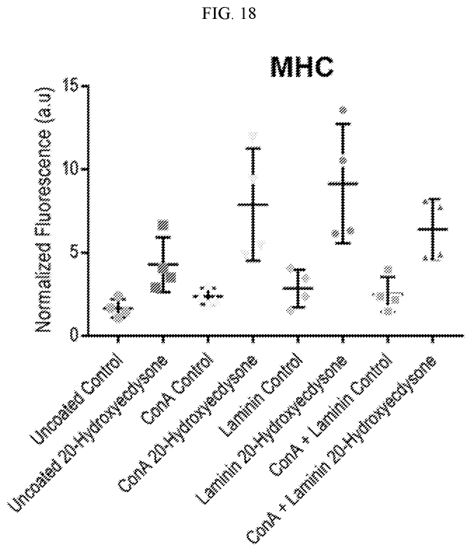

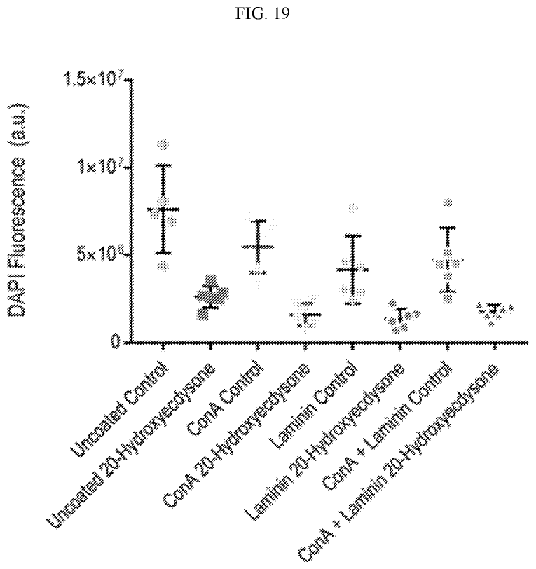

As shown in FIG. 13, supplementing growth medium (Schneider's Growth Media and 10% Fetal Bovine Serum) with sericin protein (7.5-60 ug/mL) may increase DrAMPC viability/proliferation. A subset of DrAMPCs express Connectin (a homophilic cell-cell adhesion protein expressed in a subset of muscle and neuron cells) (FIGS. 14 and 15). A subset of DrAMPCs express Neuroglian (cell adhesion protein expressed in neuron cells) (FIGS. 16 and 17).

As shown in FIGS. 18 and 19, expression of myosin heavy chain in differentiated DrAMPCs may increase in cultures coated with laminin or Concanavalin A, but the coatings do not increase cell attachment.

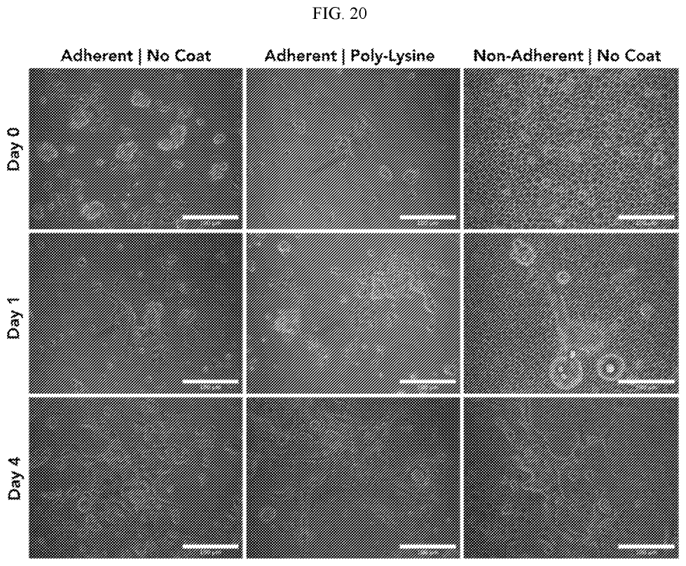

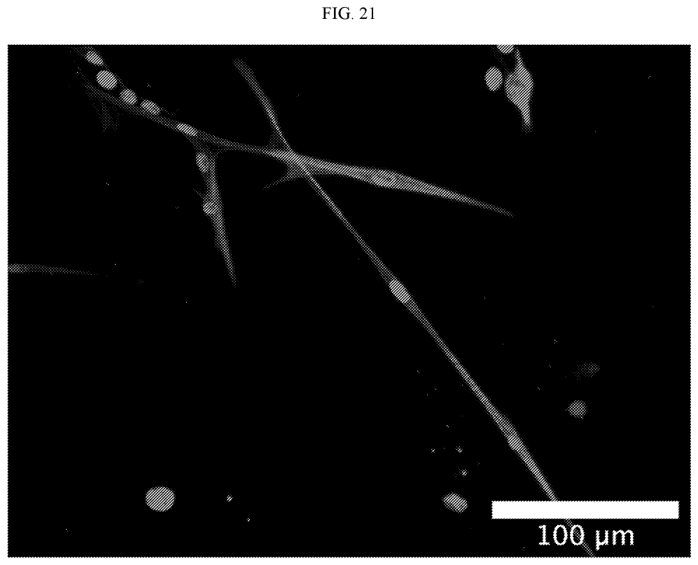

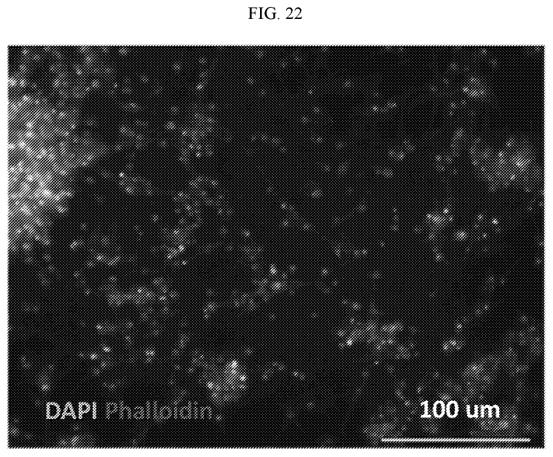

Seeding Manduca sexta embryonic precursor cell (Ms-EPCs) on poly-lysine coated plastic influences cell morphology (more cell elongation, perhaps differentiation), as shown in FIG. 20. By keeping non-adherent cell populations (e.g., yolk cells) rather than rinsing them off, larger muscle constructs are generated. Primary Ms-EPC cells can be differentiated with 20-hydroxyecdysone and are multinucleated, striated and express myosin heavy chain (FIG. 21).

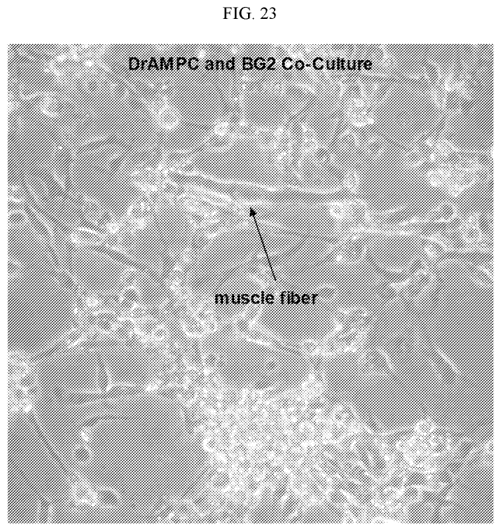

Additionally, co-culture of neuron cells with DrAMPCs may help insect muscle cells differentiate in vitro. For example, ML-DmBG2 Drosophila melanogaster central nervous system cells (BG2 cells) may co-cultured with the DrAMPCs to promote muscle cell differentiation. When co-culturing DrAMPCs with BG2 cells, muscle-like constructs begin to appear (FIG. 23).

1. A cultured meat product comprising a confluent serum-free insect muscle cell culture seeded on a food safe substrate.

2. The cultured meat product of claim 1, wherein the substrate is a film.

3. The cultured meat product of claim 1, wherein the substrate is a sponge.

4. The cultured meat product of claim 1, wherein the substrate is a chitosan substrate.

5. The cultured meat product of claim 4, wherein the chitosan substrate is a mushroom chitosan substrate.

6. The cultured meat product of claim 1, wherein the insect muscle cells are Drosophila melanogaster cells.

7. The cultured meat product of claim 1, wherein the insect muscle cells are multinucleated myotubes derived from Drosophila adult muscle progenitor cells (DrAMPCs) or Manduca sexta embryonic precursor cells (Ms-EPC).

8. The cultured meat product of claim 7, wherein the DrAMPC or Ms-EPC derived multinucleated myotubes express myosin heavy chain and ecdysone receptor.

9. The cultured meat product of claim 7, wherein the DrAMPC or Ms-EPC derived multinucleated myotubes are produced by a method comprising: culturing a population of DrAMPCs or Ms-EPCs in suspension in serum-free culture medium comprising 20-hydroxyecdysone.

10. The cultured meat product of claim 9, wherein the serum-free culture medium additionally comprises dextran sulfate, sericin, methoprene, or combinations thereof.

11. A method for producing a cultured meat product comprising the steps of:
culturing insect muscle cells on a food safe substrate in serum-free culture medium for a time sufficient for the cells to reach confluence.

12. The method of claim 11, wherein the insect muscle cells are DrAMPC or Ms-EPC derived multinucleated myotubes.

13. The method of claim 12, wherein the DrAMPC or Ms-EPC derived multinucleated myotubes express myosin heavy chain and ecdysone receptor.

14. The method of claim 12, wherein the DrAMPC or Ms-EPC derived multinucleated myotubes are produced by a method comprising: culturing a population of DrAMPCs or Ms-EPCs in suspension in serum-free culture medium comprising 20-hydroxyecdysone.

15. The method of claim 14, wherein the serum-free culture medium additionally comprises methoprene, sericin, dextran sulfate, or combinations thereof.

16. The method of claim 14, wherein prior to culturing in suspension, the DrAMPCs are cultured in serum-free culture medium on an adherent substrate.

17. The method of claim 16, wherein the adherent substrate is coated with plasma, Concanavalin A, laminin, or poly-lisine.

18. The method of claim 16, wherein the DrAMPCs are seeded on the adherent substrate at a density between about 55,000 cells/mL and about 95,000 cells/ml.

19. The method of claim 16, wherein prior to culturing in serum-free culture medium, the DrAMPCs are cultured in culture medium supplemented with serum for at least about 2 days.

20-27. (canceled)

28. A bioactuator comprising:
confluent insect muscle cells cultured in a flexible substrate to form muscle fibers, wherein a first end of the insect muscle fiber is attached to a first anchor and a second end of the insect muscle fiber is attached to a second anchor;
at least one electrode contacting the flexible substrate; and
a culture medium contacting the flexible substrate and muscle fibers.

29-33. (canceled)
